(19) United States
(12) Patent Application Publication
(10) Pub. No.: US 2024/0161914 A1
(43) Pub. Date: May 16, 2024

(54) Method, System, and Computer Program Product for Automatic Contract Management Activation

(71) Applicant: Baptist Health South Florida, Inc., Coral Gables, FL (US)

(72) Inventor: George S. Godfrey, Miami Shores, FL (US)

(21) Appl. No.: 18/510,830

(22) Filed: Nov. 16, 2023

Related U.S. Application Data
(60) Provisional application No. 63/384,039, filed on Nov. 16, 2022.

(51) Int. Cl. G16H 40/20; G06N 5/022; G06Q 10/0631; G06Q 10/0639; G06Q 50/18

(52) U.S. Cl. CPC G16H 40/20; G06N 5/022; G06Q 10/0631; G06Q 10/0639; G06Q 50/18

(57) ABSTRACT

A method, system, and computer program product for diagnosing a condition of a case of a resource planning system using at least one operating parameter, correlating, by perception nodes of a procurement inference engine, at least one operating parameter with at least one critical parameter of the resource planning system, to generate a prediction of an activity aligned with the at least one critical parameter, comparing the at least one critical parameter with a predefined threshold value to identify at least one deviation including one or more discrepancies when a critical parameter falls, and controlling a contract management activity, based on the prediction of the activity aligned with the at least one critical parameter, by generating at least one of a response, a communication, or an action of a contract management system and adapting by automatically adjusting settings or operations to eliminate the deviation to maintain optimal performance.

### CROSS-REFERENCE TO RELATED APPLICATION

This application claims the benefit of U.S. Provisional Patent Application No. 63/384,039, filed Nov. 16, 2022, the disclosure of which is hereby incorporated by reference in its entirety.

### BACKGROUND

### 1. Field

The disclosed subject matter relates generally to methods, systems, and computer program products for automatic contract management activation, and, in some particular embodiments or aspects, for activating an automatic contract management response, activating a contract management prediction, or automatically escalating communications with suppliers or manufacturers to avoid incorrect processing of health care transactions.

### 2. Technical Considerations

Within an institution's operations, various enterprise computer systems are configured and deployed to manage the processing of goods, services, invoices, agreements, and other items received from customers, suppliers, service providers, and other entities. While some suppliers still send invoices in non-digital formats, necessitating manual entry by accounts payable (AP) professionals into their Enterprise Procurement Systems (EPS)/AP software, this manual process often results in transcription errors. Alternatively, some items are presented electronically, such as invoice images or other electronic forms. However, institutions employing different computer systems might use hardware and software tools to validate and process items leading to divergent results.

As new technologies find practical applications across various domains, organizations seek new solutions to tackle new and intricate business challenges. For example, applications play a crucial role in generating essential information through activities such as record review, verification, segmentation, security checks, customer support, auditing, and record-keeping. While current EPS/Enterprise Resource Planning (ERP) systems excel at transaction creation and financial information capture, they are compromised when it comes to data management, visualization, and communication.

For example, supply chain systems heavily rely on EPS or ERP systems to manage administrative tasks. However, these systems often fall short in efficiently addressing issues and determining necessary actions due to their inability to effectively process and communicate the multitude of data points. Additionally, such critical systems lack the capacity to seamlessly implement solutions for observed issues without significant manual intervention.

Moreover, existing hardware and software tools used to identify and resolve diverging processes may be unable to correctly process items with various limitations, and when used to identify and resolve actions arising from processing of contracts, terms and conditions, and including limited accuracy and speed at which they can perform such processing.

### SUMMARY

Accordingly, it is an object of the presently disclosed subject matter to provide systems, methods, and computer program products for automatic contract management activation that overcome some or all of the deficiencies identified above.

According to non-limiting embodiments or aspects, provided is a computer-implemented method, comprising: a computer-implemented method, comprising: diagnosing a condition of a case of a resource planning system using at least one operating parameter; correlating, by one or more perception nodes of a procurement inference engine, at least one operating parameter with at least one critical parameter of the resource planning system, wherein the one or more perception nodes comprise a neural network trained to generate a prediction of an activity aligned with the at least one critical parameter; comparing the at least one critical parameter with a predefined threshold value to identify at least one deviation including one or more discrepancies when a critical parameter falls; and controlling a contract management activity, based on the prediction of the activity aligned with the at least one critical parameter, by generating at least one of a response, a communication, or an action of a contract management system and adapting by automatically adjusting settings or operations to eliminate the at least one deviation to maintain optimal performance.

According to non-limiting embodiments or aspects, provided is a system, comprising: a system, comprising: a memory; and at least one processor coupled to the memory and configured to: diagnose a condition of a case of a resource planning system using at least one operating parameter; correlate, by one or more perception nodes of a procurement inference engine, at least one operating parameter with at least one critical parameter of the resource planning system, wherein the one or more perception nodes comprises a neural network trained to correlate an activity with the at least one critical parameter; compare the at least one critical parameter with a predefined item threshold value; and control a contract management activity, based on a prediction of the activity aligned with the at least one critical parameter, by generating at least one of a response, a communication, or an action of a contract management system and adapting by automatically adjusting settings or operations to eliminate at least one deviation to maintain optimal performance.

According to non-limiting embodiments or aspects, provided is a non-transitory computer-readable medium having instructions stored thereon that, when executed by at least one computing device, cause the at least one computing device to: diagnose a condition of a case of a resource planning system using at least one operating parameter; correlate, by one or more perception nodes of a procurement inference engine, at least one operating parameter with at least one critical parameter of the resource planning system, wherein the one or more perception nodes comprises a neural network trained to correlate an activity with the at least one critical parameter; compare the at least one critical parameter with a predefined item threshold value; and control a contract management activity, based on a prediction of the activity aligned with the at least one critical parameter, by generating at least one of a response, a communication, or an action of a contract management system and adapting by automatically adjusting settings or operations to eliminate at least one deviation to maintain optimal performance.

Further non-limiting embodiments or aspects are set forth in the following numbered clauses:

Clause 1: A computer-implemented method, comprising: diagnosing a condition of a case of a resource planning system using at least one operating parameter; correlating, by one or more perception nodes of a procurement inference engine, at least one operating parameter with at least one critical parameter of the resource planning system, wherein the one or more perception nodes comprise a neural network trained to generate a prediction of an activity aligned with the at least one critical parameter; comparing the at least one critical parameter with a predefined threshold value to identify at least one deviation including one or more discrepancies when a critical parameter falls; and controlling a contract management activity, based on the prediction of the activity aligned with the at least one critical parameter, by generating at least one of a response, a communication, or an action of a contract management system and adapting by automatically adjusting settings or operations to eliminate the at least one deviation to maintain optimal performance.

Clause 2: The computer-implemented method according to clause 1, wherein the procurement inference engine includes further instructions, which cause the procurement inference engine to: diagnose a contract renewal in a first machine learning (ML) model, diagnose a contract maintenance in a second ML model, and diagnose a requisition placement in a third ML model; diagnose, by the first ML model, an action to take for a contract renewal, wherein one or more inference results are associated with at least one of an initiator, an expiration date, an owner, contract information for a response type associated with at least one of emergency preparedness, a placement, a consignment, a service, a construction, an evaluation, a group purchasing organization (GPO), or a local pricing; diagnose, by the second ML model, an action to take for a contract maintenance by determining a critical factor associated with a reason for the contract maintenance from at least one of assign to owner, cancelation, current contract, price change, performance notification; and diagnose, by the third ML model, an assignment of a requisition placement to a commodity.

Clause 3: The computer-implemented method clauses 1-2, wherein the one or more operating parameters are formed from at least one specified field and are correlated to at least one critical parameter including at least one reason for a contract renewal, a contract maintenance, or a requisition placement.

Clause 4: The computer-implemented method clauses 1-3, wherein a call to action communication is generated or configured for at least one of an intercompany action, an external action, a response management, or a resolution deployment.

Clause 5: The computer-implemented method clauses 1-4, comprising: generating or configuring a call to action based on at least one of: a critical parameter of a cost analysis correlated from operating parameters comprising information about at least one of a usage, a critical parameter of a cost, a critical parameter of an offset, a critical parameter of current product details, or a critical parameter of new product details, a critical parameter of a contract generated with one or more terms, or a critical parameter for one or more identified alternative items, wherein a critical parameter for the call to action is correlated from a plurality of operating parameters defined by one or more of a current contract identification, a contract description, a supplier name, items on a contract, a start date, an end date, a product name, an item number, a catalog number, a cost, an estimated usage per year, an annualized total cost, product information, a product domain, a product description, a product identification, an alert detail, an alert identification, a release date/type, an alert description, a supplier, an alert reason, a recall level, instructions, additional information, contact information, an inventory item identification, a description, a manufacturer item identification, a vendor item identification, a commodity contract administrator, a clinical resource liaison, a requestor, or a surgery business office manager, and wherein an agreement type includes at least one of a lease, emergency preparedness, a placement, a consignment, a service, construction, an evaluation, GPO, or local pricing.

Clause 6: The computer-implemented method of clauses 1-5, wherein the call to action comprises: generating an analysis for at least one of: one or more items of a contract renewal, one or more items of a contract maintenance, or one or more purchase requests of a requisition placement in a plurality of accounts of the resource planning system; and wherein an analysis of the contract renewal includes at least one of a cost analysis of the usage, a cost analysis of the cost, a cost analysis of an actual spend, an expected contract value (ECV), a probability of the ECV, a contract of indefinite duration, a one-time contract, a quality analysis, a recalls analysis, a rebid for consumer to consumer, or a department approval, one or more contract terms, supplier information, contact information, training determination, source information including minority owned, existing, or new, stocking information, or stocking location, wherein an analysis of a contract maintenance for an existing agreement includes at least one of an analysis of one or more calls to action of a cancelation, a price change, or a performance notification, and wherein an analysis of a requisition management for one or more products includes at least one of one or more call to actions, including one or more reason codes, one or more services, one or more items, one or more goods, an existing product, product details, or a source strategy assignment.

Clause 7: The computer-implemented method according to clauses 1-6, wherein the procurement inference engine is configured to generate an intercompany action, comprising: an intercompany action for contract renewal includes automatically generating a call to action for at least one of: a communication to an end user; a contract type, a department approval, determining a renewal, a renew action, a request for proposal, an ad hoc approver, an email generation critical parameter including an operating parameter template, or one or more attachments; an intercompany action for a contract maintenance for an existing agreement includes at least one of automatically generating a draft agreement based on predicted clauses, at least one department approval, at least one preset commodity groups, at least one ad hock approver, or a contract maintenance use template; and an intercompany action for a requisition management for one or more products includes determining when a requisition is needed, a call to action operating parameter template, or one or more attachment.

Clause 8: A system, comprising: a memory; and at least one processor coupled to the memory and configured to: diagnose a condition of a case of a resource planning system using at least one operating parameter; correlate, by one or more perception nodes of a procurement inference engine, at least one operating parameter with at least one critical parameter of the resource planning system, wherein the one or more perception nodes comprises a neural network trained to correlate an activity with the at least one critical parameter; compare the at least one critical parameter with a predefined item threshold value; and control a contract management activity, based on a prediction of the activity aligned with the at least one critical parameter, by generating at least one of a response, a communication, or an action of a contract management system and adapting by automatically adjusting settings or operations to eliminate at least one deviation to maintain optimal performance.

Clause 9: The system clause 8, wherein the procurement inference engine includes further instructions, which cause the procurement inference engine to: diagnose a contract renewal in a first machine learning (ML) model, diagnose a contract maintenance in a second ML model, and diagnose a requisition placement in a third ML model; diagnose, by the first ML model, an action to take for a contract renewal, wherein one or more inference results are associated with at least one of an initiator, an expiration date, an owner, contract information for a response type associated with at least one of emergency preparedness, a placement, a consignment, a service, a construction, an evaluation, a group purchasing organization (GPO), or a local pricing; diagnose, by the second ML model, an action to take for a contract maintenance by determining a critical factor associated with a reason for the contract maintenance from at least one of assign to owner, cancelation, current contract, price change, performance notification; and diagnose, by the third ML model, an assignment of a requisition placement to a commodity.

Clause 10: The system clauses 8-9, wherein the one or more operating parameters are formed from at least one specified field and are correlated to at least one critical parameter including at least one reason for a contract renewal, a contract maintenance, or a requisition placement.

Clause 11: The system clauses 8-10, wherein a call to action communication is generated or configured for at least one of an intercompany action, an external action, a response management, or a resolution deployment.

Clause 12: The system clauses 8-11, comprising: generating or configuring a call to action based on at least one of: a critical parameter of a cost analysis correlated from operating parameters comprising information about at least one of a usage, a critical parameter of a cost, a critical parameter of an offset, a critical parameter of current product details, or a critical parameter of new product details, a critical parameter of a contract generated with one or more terms, or a critical parameter for one or more identified alternative items, wherein a critical parameter for the call to action is correlated from a plurality of operating parameters defined by one or more of a current contract identification, a contract description, a supplier name, items on a contract, a start date, an end date, a product name, an item number, a catalog number, a cost, an estimated usage per year, an annualized total cost, product information, a product domain, a product description, a product identification, an alert detail, an alert identification, a release date/type, an alert description, a supplier, an alert reason, a recall level, instructions, additional information, contact information, an inventory item identification, a description, a manufacturer item identification, a vendor item identification, a commodity contract administrator, a clinical resource liaison, a requestor, or a surgery business office manager, and wherein an agreement type includes at least one of a lease, emergency preparedness, a placement, consignment, a service, construction, an evaluation, GPO, or local pricing.

Clause 13: The system clauses 8-12, wherein the call to action includes further instructions, which when executed cause the procurement inference engine to: generate an analysis for at least one of: one or more items of a contract renewal, one or more items of a contract maintenance, or one or more purchase requests of a requisition placement in a plurality of accounts of the resource planning system; and wherein an analysis of the contract renewal includes at least one of a cost analysis of the usage, a cost analysis of the cost, a cost analysis of an actual spend, an expected contract value (ECV), a probability of the ECV, a contract of indefinite duration, a one-time contract, a quality analysis, a recalls analysis, a rebid for consumer to consumer, or a department approval, one or more contract terms, supplier information, contact information, training determination, source information including minority owned, existing, or new, stocking information, stocking location, and/or the like, wherein an analysis of a contract maintenance for an existing agreement includes at least one of an analysis of one or more calls to action of a cancelation, a price change, or a performance notification, and wherein an analysis of a requisition management for one or more products includes at least one of one or more call to actions, including one or more reason codes, one or more services, one or more items, one or more goods, an existing product, product details, or a source strategy assignment.

Clause 14: The system clauses 8-13, wherein the procurement inference engine includes instructions to generate an intercompany action, comprising: an intercompany action for contract renewal includes automatically generating a call to action for at least one of: a communication to an end user; a contract type, a department approval, determining a renewal, a renew action, a request for proposal, an ad hoc approver, an email generation critical parameter including an operating parameter template, or one or more attachments, an intercompany action for a contract maintenance for an existing agreement includes at least one of automatically generating a draft agreement based on predicted clauses, at least one department approval, at least one preset commodity groups, at least one ad hock approver, or a contract maintenance use template; and an intercompany action for a requisition management for one or more products includes determining when a requisition is needed, a call to action operating parameter template, or one or more attachment.

Clause 15: A non-transitory computer-readable medium having instructions stored thereon that, when executed by at least one computing device, cause the at least one computing device to: diagnose a condition of a case of a resource planning system using at least one operating parameter; correlate, by one or more perception nodes of a procurement inference engine, at least one operating parameter with at least one critical parameter of the resource planning system, wherein the one or more perception nodes comprises a neural network trained to correlate an activity with the at least one critical parameter; compare the at least one critical parameter with a predefined item threshold value; and control a contract management activity, based on a prediction of the activity aligned with the at least one critical parameter, by generating at least one of a response, a communication, or an action of a contract management system and adapting by automatically adjusting settings or operations to eliminate at least one deviation to maintain optimal performance.

Clause 16: The non-transitory computer-readable medium clause 15, wherein the procurement inference engine includes further instructions, which when cause the procurement inference engine to: diagnose a contract renewal in a first machine learning (ML) model, diagnose a contract maintenance in a second ML model, and diagnose a requisition placement in a third ML model; diagnose, by the first ML model, an action to take for a contract renewal, wherein one or more inference results are associated with at least one of an initiator, an expiration date, an owner, contract information for a response type associated with at least one of emergency preparedness, a placement, a consignment, a service, a construction, an evaluation, a group purchasing organization (GPO), or a local pricing; diagnose, by the second ML model, an action to take for a contract maintenance by determining a critical factor associated with a reason for the contract maintenance from at least one of an assigned owner, cancelation, current contract, price change, performance notification; and diagnose, by the third ML model, an assignment of a requisition placement to a commodity.

Clause 17: The non-transitory computer-readable medium clauses 15-16, wherein the one or more operating parameters are formed from at least one specified field and are correlated to at least one critical parameter including at least one reason for a contract renewal, a contract maintenance, or a requisition placement.

Clause 18: The non-transitory computer-readable medium clauses 15-17, wherein a call to action communication is generated or configured for at least one of an intercompany action, an external action, a response management, or a resolution deployment.

Clause 19: The non-transitory computer-readable medium clauses 15-18, comprising: generating or configuring a call to action based on at least one of: a critical parameter of a cost analysis correlated from operating parameters comprising information about at least one of a usage, a critical parameter of a cost, a critical parameter of an offset, a critical parameter of current product details, or a critical parameter of new product details, a critical parameter of a contract generated with one or more terms, or a critical parameter for one or more identified alternative items, wherein a critical parameter for the call to action is correlated from a plurality of operating parameters defined by one or more of a current contract identification, a contract description, a supplier name, items on contract, a start date, an end date, a product name, an item number, a catalog number, a cost, an estimated usage per year, an annualized total cost, product information, a product domain, a product description, a product identification, an alert detail, an alert identification, a release date/type, an alert description, a supplier, an alert reason, a recall level, instructions, additional information, contact information, an inventory item identification, a description, a manufacturer item identification, a vendor item identification, a commodity contract administrator, a clinical resource liaison, a requestor, or a surgery business office manager, and wherein an agreement type includes at least one of a lease, emergency preparedness, a placement, a consignment, a service, construction, an evaluation, GPO, or local pricing.

Clause 20: The non-transitory computer-readable medium clauses 15-19, wherein the call to action comprises: generating an analysis for at least one of: one or more items of a contract renewal, one or more items of a contract maintenance, or one or more purchase requests of a requisition placement in a plurality of accounts of the resource planning system; and wherein an analysis of the contract renewal includes at least one of a cost analysis of the usage, a cost analysis of the cost, a cost analysis of an actual spend, an expected contract value (ECV), a probability of the ECV, a contract of indefinite duration, a one-time contract, a quality analysis, a recalls analysis, a rebid for consumer to consumer, or a department approval, one or more contract terms, supplier information, contact information, training determination, source information including minority owned, existing, or new, stocking information, or stocking location, wherein an analysis of a contract maintenance for an existing agreement includes at least one of an analysis of one or more calls to action of a cancelation, a price change, or a performance notification, and wherein an analysis of a requisition management for one or more products includes at least one of one or more call to actions, including one or more reason codes, one or more services, one or more items, one or more goods, an existing product, product details, or a source strategy assignment.

Accordingly, it is an object of the presently disclosed subject matter to provide methods, systems, and computer program products for securely rendering sensitive data.

These and other features and characteristics of the presently disclosed subject matter, as well as the methods of operation and functions of the related elements of structures and the combination of parts and economies of manufacture, will become more apparent upon consideration of the following description and the appended claims with reference to the accompanying drawings, all of which form a part of this specification, wherein such as reference numerals designate corresponding parts in the various figures. It is to be expressly understood, however, that the drawings are for the purpose of illustration and description only and are not intended as a definition of the limits of the disclosed subject matter. As used in the specification and the claims, the singular form of “a,” “an,” and “the” include plural referents unless the context clearly dictates otherwise.

### BRIEF DESCRIPTION OF THE DRAWINGS

Additional advantages and details of the disclosed subject matter are explained in greater detail below with reference to the exemplary embodiments that are illustrated in the accompanying figures, in which:

FIG. 1 is a diagram of a non-limiting embodiment of an environment in which methods, systems, and/or computer program products for automatic contract management activation, described herein, which may be implemented according to the principles of the presently disclosed subject matter;

FIG. 2 is a flow diagram of a non-limiting embodiment for automatic contract management activation;

FIG. 3 is a step diagram for a method to automate contract management activation in a supply chain;

FIG. 4 is a diagram of a non-limiting embodiment of an environment in which methods, systems, and/or computer program products, described herein, may be implemented for automatic contract management activation according to the principles of the presently disclosed subject matter;

FIG. 5 illustrates example components of a device used in connection with non-limiting embodiments; and

FIGS. 6A-6E are exemplary call-to-action illustrations for automatic contract management activation according to non-limiting embodiments.

### DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the disclosed subject matter as it is oriented in the drawing figures. However, it is to be understood that the disclosed subject matter may assume various alternative variations and step sequences, except where expressly specified to the contrary. It is also to be understood that the specific devices and processes illustrated in the attached drawings, and described in the following specification, are simply exemplary embodiments or aspects of the disclosed subject matter. Hence, specific dimensions and other physical characteristics related to the embodiments or aspects disclosed herein are not to be considered as limiting unless otherwise indicated.

No aspect, component, element, structure, act, step, function, instruction, and/or like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or like) and may be used interchangeably with “one or more” or “at least one.” Where only one item is intended, the term “one” or similar language is used. Also, as used herein, the terms “has,” “have,” “having,” or like are intended to be open-ended terms. Further, the phrase “based on” is intended to mean “based at least partially on” unless explicitly stated otherwise.

As used herein, satisfying a threshold may refer to a value (e.g., a score, a power consumption, etc.) being greater than the threshold, more than the threshold, higher than the threshold, greater than or equal to the threshold, less than the threshold, fewer than the threshold, lower than the threshold, less than or equal to the threshold, equal to the threshold, etc.

As used herein, the terms “communication” and “communicate” may refer to the reception, receipt, transmission, transfer, provision, and/or like of information (e.g., data, signals, messages, instructions, commands, and/or like). For one unit (e.g., a device, a system, a component of a device or system, combinations thereof, and/or like) to be in communication with another unit means that the one unit is able to directly or indirectly receive information from and/or transmit information to the other unit. This may refer to a direct or indirect connection (e.g., a direct communication connection, an indirect communication connection, and/or like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit when at least one intermediary unit (e.g., a third unit located between the first unit and the second unit) processes information received from the first unit and communicates the processed information to the second unit. In some non-limiting embodiments or aspects, a message may refer to a network packet (e.g., a data packet and/or like) that includes data. It will be appreciated that numerous other arrangements are possible.

As used herein, the terms “client” and “client device” may refer to one or more client-side devices or systems (e.g., remote from a service or healthcare provider) used to handle a match exception (e.g., a transaction, action, or communication in association with a call to action or other activity associated with a match exception). As an example, a “client device” may refer to one or more devices used by a vendor or supplier, one or more host computers used by a supplier or vendor, one or more mobile devices used by a user, and/or like. In some non-limiting embodiments or aspects, a client device may be an electronic device configured to communicate with one or more networks and initiate or facilitate transactions. For example, a client device may include one or more computers, portable computers, laptop computers, tablet computers, mobile devices, cellular phones, wearable devices (e.g., watches, glasses, lenses, clothing, PDAs, and/or like). Moreover, a “client” may also refer to an entity (e.g., a vendor, a supplier, and/or like) that owns, utilizes, and/or operates a client device for transactions (e.g., for transactions within a supply chain).

As used herein, the term “computing device” may refer to one or more electronic devices configured to process data. A computing device may, in some examples, include the necessary components to receive, process, and output data, such as a processor, a display, a memory, an input device, a network interface, and/or like. A computing device may be a client device or a mobile device. As an example, a mobile device may include a cellular phone (e.g., a smartphone, standard cellular phone, etc.), a portable computer, a wearable device (e.g., watches, glasses, lenses, clothing, and/or like), a personal digital assistant (PDA), and/or other such as devices. A computing device may also be a desktop computer or other form of non-mobile computer.

As used herein, the term “server” may refer to one or more computing devices (e.g., processors, storage devices, similar computer components, and/or like) that communicate with client devices and/or other computing devices over a network (e.g., a public network, the Internet, a private network, and/or like) and, in some examples, facilitate communication among other servers and/or client devices. It will be appreciated that various other arrangements are possible. As used herein, the term “system” may refer to one or more computing devices or combinations of computing devices (e.g., processors, servers, client devices, software applications, components of such, and/or like). Reference to “a device,” “a server,” “a processor,” and/or like, as used herein, may refer to a previously-recited device, server, or processor that is recited as performing a previous step or function, a different server or processor, and/or a combination of servers and/or processors. For example, as used in the specification and the claims, a first server or a first processor that is recited as performing a first step or a first function may refer to the same or different server or the same or different processor recited as performing a second step or a second function.

As used herein, the terms “enterprise resource planning” or “ERP” may refer to systems and/or software designed to manage and integrate the functions of core business processes such as finance, HR, supply chain and inventory management in a single system, including software components (e.g., interfaces, independent software deployments, etc.), each of which focuses on a distinct process, including the ERP finance component which automates basic accounting, invoicing, healthcare analysis, forecasting and reporting, into a single ERP system to manage all of the healthcare transactions and accounting for multiple healthcare units, suppliers, or patients, other ERP components include order management, customer relationship management (“CRM”), purchasing (e.g., procurement), and human resources (“HR”) to handle employee records, benefits management and payroll.

As used herein, the term “supervised learning” may refer to one or more machine learning algorithms that start with known input variables (x) and an output variable (y), and learn the mapping function from the input to the output. The goal of supervised learning is to approximate the mapping function so that predictions can be made about new input variables (x) that can be used to predict the output variables (y) for that data. The process of a supervised algorithm learning from the training dataset can be thought of as a teacher supervising the learning process. The correct answers are known. The algorithm iteratively makes predictions on the training data and is corrected by the teacher. Learning stops when the algorithm achieves an acceptable level of performance. Supervised learning problems can be further grouped into regression problems and classification problems. Supervised learning techniques can use labeled (e.g., classified) training data with normal and outlier data, but are not as reliable because of the lack of labeled outlier data. For example, multivariate probability distribution based systems are likely to score the data points with lower probabilities as outliers. A regression problem is when the output variable is a real value, such as “dollars” or “exceptions”.

An exemplary regression problem includes predicting a contract renewal date to predict a numerical value based on historical data. For example, predicting the exact renewal date for a contract based on various features, such as contract history, supplier performance, and market conditions based on historical contract data, supplier performance metrics, market data, contract details, and/or the like, to generate a numerical value representing the predicted renewal date. For example, regression algorithms like linear regression, decision trees, or random forest regression are generated to make predictions about historical data and identify patterns to forecast the renewal date more accurately. An exemplary classification problem sorts the output variables into a category, such as “red” and “blue,” or “compliant” and “non-compliant”. Such a classification problem includes categorizing purchase requisitions to assign a data point to a specific category or class. For example, categorizing purchase requisitions into groups like “urgent,” “standard,” or “low priority” based on their critical parameters, supplier performance, and internal requirements to predict details of purchase requisitions, critical parameters, supplier performance scores, internal requirements, and/or the like. A category label (e.g., “urgent,” “standard,” “low priority”, etc.) may be predicted for each purchase requisition. Classification algorithms like logistic regression, decision trees, or support vector machines can be used by learning from historical data to classify new purchase requisitions into the appropriate category based on features and attributes. Improved precise predictions and categorizations, aid in contract management, requisition placement, and overall procurement efficiency.

As used herein, the term “unsupervised learning” may refer to an algorithm which has input variables (x) and no corresponding output variables. The goal for unsupervised learning is to model the underlying structure or distribution in the data in order to learn more about the data. Unlike supervised learning, in unsupervised learning there are no correct answers and there is no teacher. Unsupervised learning algorithms are used to discover and present the interesting structure in the data. Unsupervised learning problems can be further grouped into clustering and association problems. A clustering problem is modeling used to discover the inherent groupings in a dataset, such as grouping customers by purchasing behavior. An association rule learning problem may be used to discover rules that describe large portions of data, such as supplier performance rules to discover rules that link supplier performance metrics (e.g., on-time delivery, quality, responsiveness, etc.) to contract renewal or termination decisions (e.g., when supplier performance rating is ‘excellent’ and contract is expiring in 90 days, then initiate contract renewal, etc.), cost savings association rules to identify rules that define conditions under which cost-saving opportunities are recognized (e.g., when historical cost savings exceed 10% for a specific supplier and contract, initiate a price negotiation, etc.), inventory management rules to determine rules that optimize inventory levels (e.g., when inventory level reaches the trigger point and there are open purchase requisitions for the item, automatically generate a purchase order, etc.), and/or the like. Some examples of unsupervised learning algorithms are clustering and likelihood modeling.

As used herein, the term “training” may refer to a process of analyzing training data to generate a model (e.g., create a machine learning algorithm, a prediction model, a classification model, a segmentation model, etc.). For example, a training server uses machine learning techniques to analyze the training data to generate the model, often the training data includes numerous examples so that a robust model is generated to solve a problem for many variations present in the data. In some non-limiting embodiments or aspects, generating the model (e.g., based on training data from a variety of sources) is referred to as “training the model.” The machine learning techniques include, for example, supervised and/or unsupervised techniques, such as decision trees (e.g., gradient boosted decision trees), logistic regressions, artificial neural networks (e.g., convolutional neural networks), Bayesian statistics, learning automata, hidden Markov modeling, linear classifiers, quadratic classifiers, association rule learning, and/or like. In some examples, the model includes a prediction model that is specific to a particular geographic location, a particular supplier, a particular vendor, and/or like. Such as decision trees for predicting the most effective negotiation strategies for contract terms with a particular supplier sourcing process, logistic regression estimating the likelihood of a contract exception occurring during the review and approval of terms and conditions, artificial neural networks learning patterns in historical contract documents to identify new patterns and best practices for contract creation and management, Bayesian statistics for determining the probability of delays or bottlenecks in the contract management steps based on historical data and external factors, like regulatory changes, hidden Markov modeling predicting the expected timeline for the contract stage for a specific type of procurement, linear classifiers estimating the likelihood of a specific concept progressing to the contract stage based on criteria such as cost, feasibility, and strategic importance, quadratic classifiers predicting the potential cost savings associated with a specific contract based on historical procurement data. Association rule learning associations between different concepts and their respective contract outcomes to optimize decision-making and resource allocation. Additionally, or alternatively, the prediction model may be specific to a particular user or thing (e.g., a supplier of a healthcare facility that uses medical devices, a pharmaceutical supplier, a supplier extending a voucher price, one or more receipts for a purchase order, receipts for a purchase order specified on a voucher, etc.). In other examples, a training server generates one or more prediction models (e.g., one or more C2C models, one or more purchase order segmentations, one or more voucher classifications, etc.) for one or more accounts (e.g., one or more supplier accounts, one or more vendor accounts, etc.), a particular group of customers, and/or like.

As used herein, the term “machine learning inference engine” may refer to a process of executing a model algorithm and returns an inference output. For example, an inference engine (e.g., inference server, etc.) may utilize one or more processing units (e.g., a central processing unit (CPU), general processing unit (GPU), tensor processing unit (TPU), field programmable gateway array (FPGA), an application-specific integrated circuit (ASIC), etc.) to execute the model algorithm. In some non-limiting embodiments or aspects, a processing choice for machine learning inference can have a significant impact on speed, throughput, latency, accuracy, rate of learning, energy efficiency, and rate of learning.

As used herein, the term “call-to-action” may refer to a specific configuration, programming instruction, software element, or prompt provided to a user (e.g., a vendor, a supplier, an employee, staff member, etc.), such that the user is called (e.g., informed, notified, prompted, etc.) to take a particular action to address or resolve a specific exception, issue, condition, or other item identified within a program or system, or a directive (e.g., notification, alert, message, etc.) to address an exceptional situation effectively, and in some non-limiting embodiments or aspects, requires human intervention of a program or system with a problem or issue therein. For example, when an exception is detected or an issue arises, a call-to-action may be used to alert, warn, or notify a user to take an action to address a problem or issue, or escalate a problem previously identified in a call-to-action to cause relevant personnel to be notified about a need to address the issue. The call-to-action prompts the individual to respond and take appropriate steps to resolve the problem, correct data discrepancies, and perform necessary actions to ensure smooth operations within the supply chain or other processes.

As used herein, the term “diagnosis” refers to the capability of analyzing and comprehensively assessing the operating parameters of a resource planning system, a database, a data visualization, and/or like. For example, this may entail a dynamic process of determining the multifaceted operating parameters of the resource planning system, comparing with critical and context-specific parameters necessary for the effective control and management of the supplier management system (e.g., the supply chain system, supplier sales automation system, etc.), or other related integrated systems. This correlation is achieved through analysis, matching, and rigorous checking of one or more data elements, data structures, or states of data elements, and/or the like, such analyses conducted within the distinct context of procurement or invoicing activities, such that the crux of the power to diagnose lies in the software's prowess to methodically scrutinize and evaluate the operating parameters of the resource planning system, and then extending the evaluation to detecting subtle nuances and intricate patterns that might otherwise go unnoticed. Thereby unveiling issues, anomalies, or complexities embedded within the system that have the potential to propagate throughout the supply chain and impede the seamless flow of procurement processes. This provides capability to pinpoint discrepancies, irregularities, initiate proactive measures, and/or the like. By proactively addressing these identified issues, the diagnosis contributes to the fortification of the supplier chain's integrity (e.g., one or more systems or processes of the supply chain, etc.) and the enhancement of the overall supply chain's resilience, wherein software-driven inferences empower organizations to preemptively mitigate disruptions, optimize operational efficiencies, and ensure the uninterrupted flow of procurement activities. The problem that the diagnosis aims to address is the effective management and optimization of the supply chain and procurement activities. By diagnosing the operating parameters and correlating them with critical parameters, potential exceptions, inefficiencies, or deviations from expected performance, issues or possible issues such as delayed orders, incorrect pricing, discrepancies in supply and demand, other irregularities that may disrupt the supply chain or procurement workflow, and may be avoided or corrected. Corrective action may involve initiating the creation of new purchase orders, reordering items, balancing transaction costs back to the suppliers, restocking inventory, or recalling defective products. A procurement prediction engine 102 may also trigger automated responses or escalations to relevant personnel or systems to resolve the identified problems and prevent any adverse impacts on the supply chain or procurement processes.

As used herein, the term “operating parameters” refers to the predefined and adjustable settings or conditions within the contract management system that dictate how the system functions, such operating parameters may include specific rules, thresholds, criterion, and/or the like, which guide the automated processes, workflows, and activations within the system. Adjusting these operating parameters allows for customization and optimization of the contract management processes based on the unique needs and requirements of the organization.

As used herein, the term “critical parameters” refers to parameters that hold significant importance due to their direct impact on the efficiency, accuracy, and effectiveness of the contract management system. Critical parameters are crucial for the proper functioning and effectiveness of the system. Adjusting critical parameters may have a substantial influence on organizational goals, how the system identifies, communicates, resolves issues within the contract management processes, and/or the like. Managing and configuring critical parameters to provide optimal performance and outcomes for contract management activities.

As used herein, the term “supply chain” refers to the series of activities and steps involved in the creation, production, distribution, and management of goods and services from their source to the end consumer. These processes encompass various stages, such as procurement, manufacturing, transportation, distribution, and ultimately delivering products to customers. In the context of the discussion above, supply chain processes specifically relate to how healthcare providers manage the flow of materials, equipment, and services needed to support patient care within a healthcare setting. These processes involve activities such as procurement, inventory management, demand forecasting, supplier relationships, logistics, and/or the like. The goal of effective supply chain management is to ensure that the right products and services are available at the right time, in the right quantity, and at the right cost. In addition, supply chain refers to the interconnected network of organizations, individuals, activities, information, resources, and technologies involved in the creation, production, distribution, and delivery of goods or services to end consumers. It encompasses the entire journey a product or service takes from its raw material stage through various stages of processing, manufacturing, transportation, storage, and ultimately reaching the end user. Supply chains can be complex, involving suppliers, manufacturers, distributors, retailers, and various intermediaries, all collaborating to ensure that products or services are efficiently produced, transported, and made available to customers in a timely and cost-effective manner. Further, the term “healthcare supply chain” may refer more specifically to monitoring and supporting the flow of medicines, medical supplies and equipment, and medical services from manufacturer to patient, including supply chain quality management and planning, supply chain automation and optimization, and supplier relationship and risk management. “Healthcare supply chain system” may refer to digital tools and technology to carry out healthcare supply chain management, and may involve using actionable inferences obtained from multi-sourced data to continuously adjust and optimize supply chain systems and processes. As used herein, the supply chain framework (e.g., supply chain network, etc.) refers to the structured approach and systematic arrangement of processes, activities, entities, and technologies involved in the production, distribution, and delivery of goods or services from suppliers to consumers. The supply chain framework includes the entire supply chain (e.g., a chain of activity that bridges multiple entities for providing products or services, etc.) from raw material acquisition through production, distribution, and ultimately to deliverables for users (e.g., the end-users, customers, etc.). The supply chain framework is optimized for efficiency, to minimize costs, to enhance collaboration, and for timely delivery. Moreover, key components of a typical supply chain framework include suppliers (e.g., One or more entities or companies that provide the raw materials, components, or services needed for production, etc.), manufacturing/production (e.g., one or more phases for transforming raw materials into finished products through various processes, etc.), distribution and logistics (e.g., movement of products from manufacturing facilities to distribution centers, warehouses, retailers, consumers, etc.), including transportation, inventory management, and order fulfillment, retailers/wholesalers (e.g., intermediaries that store and sell products to consumers or other businesses, etc.), consumers (e.g., one or more end-users or final recipients of the products or services, etc.), technology and information system (e.g., advanced technologies, software, and information systems play a crucial role in tracking and managing various aspects of the supply chain, from inventory levels to demand forecasting, communication and collaboration (e.g., communication and collaboration among all stakeholders in the supply chain for operations and timely decision-making, etc.), risk management (e.g., assessing and managing risks associated with disruptions, etc.) of supply shortages, transportation delays, and/or the like for maintaining a supply chain, sustainability and ethics (e.g., modern supply chain frameworks with sustainability practices, including ethical sourcing, environmental considerations, and social responsibility, etc.), data analytics and optimization (e.g., identifying data collected throughout the supply chain to optimize processes, improve efficiency, enhance customer satisfaction, etc.). In some non-limiting embodiments or aspects, the supply chain framework aims to create a seamless flow of goods and services while minimizing costs, reducing waste, and responding effectively to changing market conditions.

As used herein, the term “Activation” refers to the initiation or triggering of a specific process or action within the procurement inference engine or the broader supplier management system. Including triggering of the commencement of a predefined sequence of operations based on certain conditions, events, or criteria being met. In this context, activation signifies the beginning of the automated steps or procedures executed by the procurement inference engine to respond to various situations related to item management, purchasing, supplier interactions, or supplier management. As used herein, “to activate” refers to setting in motion or starting the predefined sequence of actions within a procurement inference engine or supplier management system. This may involve triggering a specific response or series of operations within the system based on identified criteria or events. Activation involves the initiation of automated processes, such as sending notifications, requesting approvals, escalating responses, or generating follow-up actions, to address issues or progress through the stages of supplier-related tasks or procurement workflows.

As used herein, the term a “concept” refers to the initial idea, need, or requirement within an organization that may lead to the creation of a procurement or purchasing agreement. It's the starting point for a process that eventually results in a formal contract. The “Concept to Contract” process includes various stages, including the development of the idea or concept, its evaluation, strategic planning, negotiation, and ultimately the creation and management of the contract itself. The concept is the first step in this procurement lifecycle.

As used herein, the term a “match exception” refers to cases within the supply chain system where there is a discrepancy (e.g., inaccuracy, misalignment, etc.) between different sets of data or information that are expected to match or correspond. These discrepancies may involve various elements, such as purchase orders (e.g., requisitions, procurement orders, acquisition requests, etc.), invoices (e.g., bills, statements, payment requests, invoices, etc.), payment amounts (e.g., transaction values, billing amounts, monetary figures, etc.), vouchers (e.g., coupons, tokens, certificates, credits, etc.), costs (e.g., expenses, expenditures, outlays, charges, etc.), other related transactional details (e.g., additional relevant transaction information, associated financial particulars, etc.). As an example, match exception refers to a difference between the information presented in an invoice and the corresponding purchase order(s). For example, when a purchase order indicates a certain cost for a healthcare product, but the associated invoice specifies a different payment amount, it may result in a match exception. Similarly, discrepancies between different purchase orders for the same item may also trigger match exceptions. Further examples are discussed below. A match exception in this context signifies a divergence or inconsistency between expected and actual data values, particularly related to purchase orders, invoices, and payments, which the procurement prediction engine disclosed herein aims to predict and address for improved efficiency and accuracy in supply chain operations.

As used herein, the term a “contract renewal” refers to refers to the process of extending or continuing an existing contractual agreement between two or more parties after its initial term has expired. During a contract renewal, the parties involved typically review the terms and conditions of the existing contract, negotiate any necessary changes or updates, and then formally agree to extend the contract for a specified period. Contract renewals are common in various business contexts, including vendor agreements, service contracts, lease agreements, and more.

As used herein, the term a “contract maintenance” refers to the ongoing management and oversight of an existing contract throughout its active term. This ongoing management and oversight of an existing contract includes activities such as monitoring compliance with contract terms, tracking performance metrics, ensuring that obligations are met by all parties, addressing any issues or disputes that may arise during the contract's lifecycle, and/or the like. Effective contract maintenance helps to safeguard the interests of the parties involved and ensures that the contract remains in force and productive.

As used herein, the term a “requisition placement” refers to the act of formally requesting and initiating the procurement or purchase of goods, services, or materials within an organization (e.g., placing a requisition). A requisition is a document or request that specifies the details of what is needed, such as the quantity, description, quality, other relevant information, and/or the like. Requisition placement is typically the first step in the procurement process, and it may trigger subsequent actions, including approval workflows, purchasing orders, internal activities, supplier communication, and/or the like to fulfill the requested items or services for efficient procurement and inventory management.

The systems and method described herein are described in the context of healthcare supply chains, but may also apply to a wide range of other industries and supply chains as well. Some examples include pharmaceuticals, similar to healthcare pharmaceutical supply chains that deal with the distribution of medications and may help in tracking and authenticating pharmaceuticals, predicting demand, and ensuring compliance with regulations. Retail, where products are sourced, managed, and distributed through various channels. The system may help in managing inventory, predicting demand, optimizing logistics, and enhancing communication with suppliers. Manufacturing, where raw materials, production processes, and distribution of finished goods help in predicting maintenance needs, optimizing production schedules, and ensuring efficient utilization of resources. Automotive, which includes complex networks of suppliers providing components for vehicle assembly help in predicting component shortages, optimizing production to meet demand, and improving collaboration with suppliers. Electronics, which includes the sourcing and assembly of electronic components and may help in managing component availability, predicting market trends, and improving communication among stakeholders. Agriculture, which includes the production, distribution, and sale of agricultural products, may help predict crop yields, optimize distribution routes, and enhance collaboration between farmers, distributors, and retailers. Food and beverage, which includes the sourcing, processing, and distribution of perishable goods, may help in managing inventory, predicting demand, and improving traceability. Logistics and transportation includes the movement of goods from suppliers to consumers and may assist by optimizing routes, predicting shipping delays, and enhancing real-time tracking. Energy includes the production and distribution of energy resources and may help in predicting maintenance needs for infrastructure, optimizing distribution networks, and managing resource availability. Textiles and apparel includes the production and distribution of clothing and textiles and may help in managing inventory, predicting fashion trends, and optimizing production schedules.

In existing systems for contract management, issues arise that require input, validation, and approvals from various stakeholders for resolution. However, these systems may have deficiencies that hinder their efficiency, accuracy, and effectiveness. For example, in critical contract management activities where structured communication is essential for collaboration, heavy reliance on manual communication (e.g., emails, phone calls, and meetings) introduces a host of inefficiencies, inaccuracies, delays, etc. The complexity of contract management processes, coupled with the diverse systems and tools in use, further compounds the inefficiencies of manual communication. Existing systems may not accurately relay information, track issue statuses, and may not provide consistent resolutions across systems and platforms, such as between parties to a contract.

Furthermore, the configuration of existing contract management communication delays a timely resolution of matters, an important aspect in contract management activities that demand quick decision-making. Communication processes may operate with a risk of miscommunication and errors. Delays in accurately relaying information among stakeholders may lead to misinterpretations, misunderstandings, or critical details being omitted. In such existing systems, miscommunications have the potential to trigger incorrect actions or decisions, with consequences that impede the overall contract management.

Another major drawback lies in the difficulty of tracking and ensuring accountability within these manual communication methods. The lack of transparency and automation makes it difficult to monitor the progress of issue resolution, leading to uncertainties about responsibilities and accountabilities. Consequently, issues might fall through the cracks or experience delays due to the absence of a streamlined process.

The lack of standardization in communication practices in existing systems, such as, for example, across different contract management functions or departments poses yet another challenge. With different stakeholders adopting varying approaches to handling similar issues, best practices and optimizing processes is neglected. Existing systems may not include automated messaging system capable of efficiently handling contract management actions, insufficient for this critical process. Such existing systems are insufficient to extend beyond communication. Existing contract management lack efficient messaging systems. This significantly impacts the resolution and dispatch of activities.

Moreover, within these systems, structured email communication heavily relies on manual interventions, introducing further inefficiencies when managing critical activities within contract management. Transmissions of contract management requests and updates to both internal and external stakeholders may be inaccurate, or repetitious.

Additionally, existing systems often provide inefficient tracking of contract management workflows, may fail to offer interface control for external systems linked to contract resolution actions, and may not maintain sufficient accuracy, which in turn leads to errors and inconsistencies in contract management. This is in contrast to a manual approach to handling contract issues, which may take a significantly longer time to even notice a contract issue. By identifying and addressing exceptions quickly through the systems and methods provided herein, the likelihood of repeating mistakes is reduced, which is a key advantage of the automated approach.

The lack of advanced analytics in existing systems hampers the classification of contract actions into targeted workflows, while also hindering the identification of suitable recipients, impacting overall responsiveness.

Furthermore, the inability to transform contract exceptions into actionable inferences limits the activation of automated workflows and navigations of external systems during resolutions.

In terms of contract management, existing systems lack the necessary automation, leading to intensive interventions that cause delays, errors, and disputes with stakeholders. Delays in resolving contract exceptions, inefficient contract approvals and processing, and inaccurate predictions further compound the inefficiencies. Further, in a manual method, when a dispute arises, users may not effectively track each of the communications that occur during the resolution process.

Existing systems ultimately struggle to accurately identify contract actions and determine suitable activities and response for approval processes. These inherent inefficiencies result in prolonged processing times and inadequate fulfillment of contract requests, thereby extending the challenges faced by organizations engaging in contract management.

Although individual team members can query and manipulate raw data from existing systems, what is needed is a unified platform that aggregates, organizes, and visualizes data daily, while also automating provision of actions and responses that are crucial to contract management. The limitations of current systems across these critical activities hinder operational efficiency, accuracy, and speed within contract management. Dynamic transformation of contract management is needed for generation of meaningful inferences by the platform. Automated actions, responses, and resolutions are needed for improvements to contract management communication process involving internal and external stakeholders. An automated solution is needed to streamline this communication process, leveraging dynamic activities for resolving issues.

Provided herein are improved methods, systems, and computer program products for efficient generation, communication and resolution of contract exceptions. For example, according to the methods, systems, and computer program products described herein, a contract management system to efficiently resolve contract discrepancies is provided. The contract management system streamlines processes by providing contract workflows for issue resolution. Contract automation provides improvements to ensure relevant stakeholders are notified promptly, approvals are obtained efficiently, actions are taken according to predefined rules, and/or the like. Contract management automation minimizes the risk of errors, accelerates response times, and improves the overall accuracy of contract management processes and related communications. Furthermore, utilizing an automated case creation approach allows for immediate deployment of call-to-action when an exception occurs.

In this way, non-limiting embodiments or aspects of the present disclosure provide an improved approach to contract management. The contract management system transforms contract exception resolution by optimizing the speed, accuracy, and efficiency of issue resolution across contract management. The approach streamlines contract management by integrating contract workflows with dynamic issue resolution. With contract automation, stakeholders receive prompt notifications, approvals are efficiently obtained, and actions adhere to predefined rules. By reducing the risk of errors, accelerating response times, and enhancing the accuracy of contract management processes and associated communications, this improved contract management system improves contract exception resolution efficiency across the entire contract management landscape.

The advantages of automating critical contract management activities are programmed or configured to improve the depth of inferences, improve predictive capabilities, and improve trend analysis crucial for high-performing contract management systems and processes. These improvements underscore the demand for innovative solutions that provide a proven, repeatable, and prescriptive process. Further advantages of automation for critical contract management activities, include the substantial reduction in time from automatic response and escalation, elimination of significant contract management activity processing, elimination of time intensive manual intervention, and/or significantly mitigating the errors that can arise from human oversight. The contract management systems provided herein, streamline and optimize workflows, ensuring smooth progress across various stages and systems.

Moreover, the capability for real-time monitoring of processes and transactions, exemplified by the contract management system, enables rapid identification and resolution of issues. By incorporating advanced analytics and machine learning algorithms, the contract management system facilitates predictive trend analysis and enhances decision-making. The new approach also ensures consistent processes throughout the contract management landscape, promoting uniform application of assessment criteria. Further, swift identification and handling of activities are core capabilities of the system, triggering predefined actions or notifications for efficient contract exception resolution.

The contract management system maintains a comprehensive record of each communication, including time stamps, that takes place during the dispute resolution process. This enhanced tracking and documentation streamlines dispute resolution and ensures a clear record of communication, contributing to more efficient and accountable processes.

The improved contract management system effectively translates vast datasets into actionable inferences, providing improved automatic activities that may be guided by concrete data rather than assumptions. Through the automation of manual tasks, the system significantly reduces the administrative workload, freeing up valuable resources for more high-priority tasks, particularly critical in activities involving the processing of numerous requests. The contract management system also improves the quality level of the diagnosis process and communication process. The systems are inherently equipped to manage a substantial volume of transactions and processes, crucial for the complex and high-volume activities characteristic of contract management.

Further improvements may involve adapting contract management for alignment with evolving regulatory requirements and industry trends. The contract management system provides a significant advancement in critical contract management activities. By addressing limitations inherent in existing systems and offering improvements for efficiency, precision, real-time inferences, predictive capabilities, and standardized processes, the improved systems described herein provide centralized communication that simplifies the process of providing resources such as assistance, escalation of issues, and conducting audits.

In this way, the contract management system addresses inherent inefficiencies and challenges. By introducing automated activations of actions, standardized communication processes, and predictive capabilities, the system streamlines critical activities within healthcare supply chains. A match exception system, integrated into the contract management framework, significantly reduces manual interventions, mitigates errors, and accelerates response times. Further improvements provide improvements in the form of real-time monitoring, advanced analytics, and machine learning algorithms for predictive trend detection and informed decision-making. The system maintains a comprehensive record of communications, streamlining dispute resolution and ensuring accountability. Moreover, by translating vast datasets into actionable inferences, it optimizes the handling of a substantial volume of transactions, crucial for the complexity of healthcare supply chains. Accordingly, these improvements result in increased efficiency, precision, and adaptability marking a substantial advancement in procurement and critical healthcare supply chain activities.

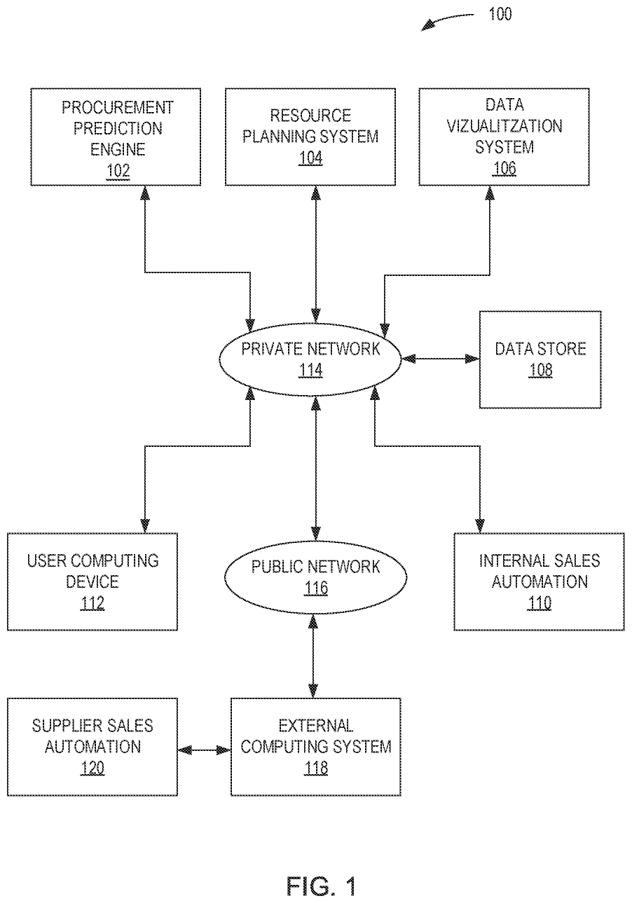

FIG. 1 shows an illustrative computing environment for automatic contract management activation processing using cognitive automation tools in accordance with one or more example embodiments. With reference to FIG. 1, computing environment 100 may include one or more computer systems. For example, computing environment 100 may include procurement prediction engine 102 (e.g., a procurement prediction platform, procurement prediction system, one or more devices of procurement prediction engine 102, etc.), resource planning system 104, data visualization system 106, data store 108, user computing device 112, internal sales automation 110, private network 114, public network 116, external computing system 118, and supplier sales automation 120.

In some non-limiting embodiments or aspects, procurement prediction engine 102 may include one or more computing devices configured to perform one or more functions for sourcing of concepts to contract as described herein. Procurement prediction engine 102 may include one or more computers (e.g., laptop computers, desktop computers, servers, server blades, etc.) that provide integration and communication among the various systems within the supply chain. In addition, procurement prediction engine 102 activates (e.g., determines, forecasts, predicts, etc.) one or more responses (e.g., reactions or actions taken in reply to specific events or triggers within the contract management system, including how stakeholders react to notifications, requests for approvals, or any other triggers within the system, and a part of the interactive and dynamic nature of managing contracts efficiently, etc.) or actions to complete activities that occur within the healthcare supply chain.

Procurement prediction engine 102 generates or invokes specific prediction models that are customized to various categories of accounts, suppliers, or transactions within its operational domain. Procurement prediction engine 102 determines one or more solutions from diverse datasets associated with different entities and activities. As a result, procurement prediction engine 102 may generate or provide distinct models that capture the unique characteristics, patterns, and behaviors relevant to each type of account, supplier, or transaction.

In some non-limiting embodiments or aspects, procurement prediction engine 102 utilizes both supervised and unsupervised learning techniques as integral components of its operational methodology. For example, procurement prediction engine 102 learns one or more relationships (e.g., features, characteristics, etc.) between input data and corresponding desired outputs, allowing it to make predictions based on this learned knowledge and also learn from the inherent structure of the data, without predetermined labels, to uncover hidden inferences or anomalies that might not be apparent through other approaches. By incorporating machine learning techniques, procurement prediction engine 102 obtains a comprehensive understanding of the data it processes. Procurement prediction engine 102 may obtain or receive labeled data of resource planning system 104 to make accurate predictions in known scenarios. Procurement prediction engine 102 may determine novel patterns for handling activities within the healthcare supply chain (e.g., a combined approach to identify a wide range of potential match exceptions, etc.). Procurement prediction engine 102 encompasses functions for generating prediction models specific to various accounts, suppliers, based on training data using machine learning techniques or transactions, performing supervised and unsupervised learning, predicting responses or actions, providing efficient integration and communication between supply chain systems, and/or the like.

In some non-limiting embodiments or aspects, resource planning system 104 may include one or more computing devices and/or other computer components (e.g., processors, memories, communication interfaces). In addition, and as illustrated in greater detail below, resource planning system 104 may be configured to interact with and/or otherwise communicate with one or more computing devices and/or other devices (e.g., customer computing devices, healthcare center computing devices, sales force automation computing devices, etc.) which may receive and/or process items (e.g., purchase orders, vouchers, agreements, etc.) being presented for payment at various locations and/or via various channels. Resource planning system 104 encompasses functions such as automating order processing across a general ledger with multiple accounts, monitoring and intercepting invoices, purchase orders, and transaction records, capturing payment indicators, costs, and exceptions associated with healthcare transactions, managing and accounting for medical inventory, including high-value and low-value items, tracking and managing inventory from value analysis to delivery through to usage, processing data related to usage for inventory management and procurement decisions, managing and integrating core business processes (finance, HR, supplier and inventory management) in a single system, automating basic accounting, invoicing, healthcare analysis, forecasting, and reporting through the ERP finance component, managing healthcare transactions and accounting for multiple healthcare units, suppliers, or patients, and handling order management, CRM, purchasing, and HR functions. Moreover, resource planning system 104 also handles employee records, benefits management, and payroll through the ERP HR component.

In some non-limiting embodiments or aspects, resource planning system 104 automates order processing across a general ledger with multiple accounts. For example, resource planning system 104 monitors and intercepts invoices, purchase orders, and transaction records. Resource planning system 104 may capture (e.g., determine, etc.) one or more payment indicators, costs, or exceptions tied to healthcare transactions. For example, resource planning system 104 manages and accounts for medical inventory, covering items of both high and low value. Resource planning system 104 tracks and manages inventory throughout its lifecycle, from value analysis to delivery and usage. Resource planning system 104 processes usage-related data to aid in inventory management and procurement decisions.

In some non-limiting embodiments or aspects, resource planning system 104 integrates and manages core business processes (e.g., finance, HR, supplier, and inventory management) within a single system. For example, resource planning system 104 may combine and oversee essential functions and activities related to various fundamental aspects of the business. These aspects include financial management, HR, supplier relationships, and inventory management. The system serves as a centralized platform where these critical operations are coordinated, synchronized, and streamlined, enhancing efficiency and facilitating smoother interactions between different components of the organization.

Further, resource planning system 104 automates basic accounting, invoicing, healthcare analysis, forecasting, and reporting through the ERP finance component. For example, resource planning system 104 includes functionalities that enable the automation of essential financial and administrative tasks. As an example, resource planning system 104 performs steps including managing financial records, generating invoices, conducting analysis related to healthcare operations, making forecasts, and generating reports. This automation is facilitated through the use of an ERP finance component, providing financial management within the broader ERP system. By automating these tasks, the system streamlines operations, reduces manual effort, enhances accuracy, and supports effective decision-making processes within the organization's financial and operational activities. Furthermore, the system pulls vendor contact information from the accounts payable (AP) data. This information is sourced from an approved vendor list, which includes relevant contact details. This ensures that the system has accurate and up-to-date vendor contact information for communication related to exception resolution and other related activities.

Resource planning system 104 handles healthcare transactions and accounting across multiple units, suppliers, and human resources (HR). For example, resource planning system 104 is responsible for managing and overseeing various aspects of financial transactions and accounting within the healthcare domain. This includes activities related to different healthcare units, suppliers providing goods or services to those units, and patients who are recipients of healthcare services. The system integrates supply chain management (SCM), AP, and HR data. HR data is used to maintain an up-to-date list of employees within the organization, which helps in identifying internal recipients of emails and ensures accuracy even when someone leaves the company. AP data, which includes information about AP processes, is used to determine the nature of the concept and how they are related to the contract (e.g., impact on the contract negotiation, the formation, the changes, etc.).

Resource planning system 104 is configured to track and record financial transactions occurring between different healthcare units, ensuring accurate and transparent accounting. It also facilitates the management of supplier-related transactions (e.g., processing invoices, purchase orders, payment records, etc.). Furthermore, the system plays a role in handling financial aspects associated with patient services, by determining billing, invoicing, and record-keeping related to patient care. In this way, resource planning system 104 handles transactions and accounting across elements of the healthcare ecosystem and thereby, contributes to efficient financial management, accurate record-keeping, and seamless coordination within the healthcare organization.

In some non-limiting embodiments or aspects, data visualization system 106 provides inferences from visual representations based on data processed by procurement prediction engine 102. In such an example, data visualization system 106 is configured to interpret and present information derived from procurement prediction engine 102 in a visual and easily understandable format. As the procurement prediction platform processes complex data and performs various predictive analyses, data visualization system 106 takes the outcomes of these analyses and translates them into visual displays, charts, graphs, and other visual representations. In some examples, visuals allow users to quickly grasp trends, patterns, and anomalies within the healthcare supply chain, enabling them to make informed decisions and take appropriate actions based on the inferences derived from the data.

As an example of an efficiency, data visualization system 106 enhances the overall usability of procurement prediction engine 102 by converting processed data and predictions into visually accessible forms (e.g. visual interface, etc.). This visual interface aids users in comprehending the information more effectively and leveraging it to optimize their SCM strategies.

Resource planning system 104 manages order processing, CRM, purchasing, and HR functions by overseeing and coordinating various elements of the organization's operations. Specifically, resource planning system 104 includes functions related to order processing, which may include the entire workflow (or portions of the workflow), for receiving, fulfilling, and managing customer orders. It also handles CRM tasks, which include managing interactions with customers, maintaining customer records, and ensuring positive customer experiences. Additionally, the system takes care of purchasing activities, including procurement of goods and services needed for the organization's operations. Lastly, resource planning system 104 plays a role in managing HR functions, which may include tasks (e.g., employee records, benefits management, payroll processing, etc.). In this way, resource planning system 104 may streamline and integrate one or more diverse functions into a cohesive overarching system. This integration means that information flows seamlessly across platforms (e.g., between one or more functions of one or more systems of computing environment 100, etc.), thereby generating accurate and up-to-date data sharing and reducing the need for manual data entry. For example, when an order is processed, the system can automatically update inventory levels, trigger procurement processes, and notify relevant departments across an enterprise, enhancing the organization's overall operational efficiency. By centralizing these functions within a single system, resource planning system 104 ensures that various departments and teams have access to consistent and accurate data, leading to better decision-making, improved coordination, and ultimately, enhanced efficiency throughout the organization's processes.

In addition, by linking the ERP with CRM, this allows auto case create and auto case close. For example, when the ERP system is connected or integrated with the customer relationship management system, it enables the automation of creating and closing cases. The integration allows for automatic creation of such cases based on certain triggers or conditions, and also the ability to automatically close these cases once they are resolved.

In some non-limiting embodiments or aspects, resource planning system 104 provides order processing across a general ledger with multiple accounts. Procurement prediction engine 102 determines or predicts one or more match exceptions based on information associated with problems caused by streamlining the processing of orders across various accounts, thereby ensuring accurate and timely execution of transactions while reducing the chances of errors and discrepancies that can lead to exceptions.

In some non-limiting embodiments or aspects, resource planning system 104 monitors and intercepts invoices, purchase orders, and transaction records. Procurement prediction engine 102 determines or predicts one or more match exceptions based on information associated with various transaction-related documents, to identify discrepancies associated with one or more match exceptions, allowing for timely intervention and resolution.

In some non-limiting embodiments or aspects, resource planning system 104 captures payment indicators, costs, and exceptions associated with healthcare transactions. Procurement prediction engine 102 determines or predicts one or more match exceptions based on information associated with capturing critical data related to payments, costs, and potential exceptions within healthcare transactions, resulting in accurate capture of information essential for identifying anomalies resulting in match exceptions.

In some non-limiting embodiments or aspects, resource planning system 104 manages or accounts for medical inventory, including high-value and low-value items. Procurement prediction engine 102 determines or predicts one or more match exceptions based on information associated with effective inventory management, including determining matching exceptions for items of varying values to avoid shortages, overages, and discrepancies that can result in exceptions.

In some non-limiting embodiments or aspects, resource planning system 104 tracks or manages inventory, for example during value analysis, delivery, usage, and/or the like, so that the supply chain remains transparent. Procurement prediction engine 102 determines or predicts one or more match exceptions based on information associated with continuous tracking of inventory, so that anomalies can be detected promptly.

In some non-limiting embodiments or aspects, resource planning system 104 processes data related to usage for inventory management and procurement decisions. Procurement prediction engine 102 determines or predicts one or more match exceptions based on information associated with stored decisions regarding inventory management and procurement, thereby minimizing the likelihood of exceptions caused by incorrect data interpretation.

In some non-limiting embodiments or aspects, resource planning system 104 manages or integrates core business processes (finance, HR, supplier and inventory management, etc.) in a single system. Procurement prediction engine 102 determines or predicts one or more match exceptions based on information associated with the integration of one or more core processes to ensure a holistic view of operations, thereby enabling faster detection and resolution of anomalies that may trigger exceptions.

In some non-limiting embodiments or aspects, resource planning system 104 automates basic accounting, invoicing, healthcare analysis, forecasting, and reporting (ERP finance component). Procurement prediction engine 102 determines or predicts one or more match exceptions based on information associated with one or more automated processes to maintain accuracy and consistency across various financial tasks, which in turn reduces the risk of exceptions due to human error.

In some non-limiting embodiments or aspects, resource planning system 104 manages healthcare transactions and accounts for multiple healthcare units, suppliers, or patients. Procurement prediction engine 102 determines or predicts one or more match exceptions based on information associated with management of transactions involving multiple entities, such that discrepancies and exceptions are quickly addressed, maintaining the integrity of the supply chain.

In some non-limiting embodiments or aspects, resource planning system 104 handles order management, CRM, purchasing, and HR functions. Procurement prediction engine 102 determines match exceptions, or the likelihood of match exceptions, arising from miscommunication or inefficiencies based on one or more problems associated with functions supporting interactions among various stakeholders.

In some non-limiting embodiments or aspects, resource planning system 104 operates in an interconnected environment that leverages external systems and networks for supplier management. Procurement prediction engine 102 leverages the interconnected environment and seamless communication with suppliers, allowing for timely sharing of information and reduced chances of exceptions due to communication gaps.

Resource planning system 104 manages employee records, benefits, and payroll through the ERP HR component to ensure that employees are paid accurately and on-time, while accounting for factors (e.g., hours worked, deductions, taxes, other applicable variables, etc.). More specifically, the system plays a role in managing employee records, which includes maintaining and updating information about employees (e.g., personal details, job roles, performance evaluations, and any changes in their employment status, or in some examples, may also include keeping track of historical data related to employees' roles and responsibilities, etc.). Furthermore, resource planning system 104 is responsible for managing employee benefits, which may include handling various benefits offered to employees (e.g., health insurance, retirement plans, paid time off, other perks, etc.). The system ensures accurate administration and distribution of these benefits, making sure that employees receive their entitled benefits in a timely manner. Additionally, resource planning system 104 manages payroll through the ERP HR component. This entails automating the process of calculating and disbursing employee salaries, wages, and other compensation. The integration of these HR functions within resource planning system 104 streamlines HR processes, reduces the chances of errors, and enhances efficiency by eliminating the need for manual handling of employee-related data and payroll calculations. This integrated approach also enables better data accuracy or compliance with regulations for employee-related matters.

In some non-limiting embodiments or aspects, data visualization system 106 includes one or more computing devices and/or other computer components (e.g., processors, memories, communication interfaces). In addition, data visualization system 106 may be configured to interact with and/or otherwise communicate with one or more computing devices and/or other devices (e.g., enterprise resource systems, database management systems, workflow systems, sales automation systems, etc.) which may receive and/or process accounts and associated payment information. Data visualization system 106 is responsible for interacting with enterprise resource systems, database management systems, workflow systems, and sales automation systems, presenting accounts and associated payment information in a visual and understandable format, providing inferences and visual representations based on data, and enhancing value analysis and item management through predictive actions and interpretations.

In some non-limiting embodiments or aspects, data visualization system 106 interfaces with various components of computing environment 100. Data visualization system 106 may provide a comprehensive view of data. For example, a visualization of the data from multiple sources of computing environment 100 may provide a holistic understanding of the supply chain.

Data visualization system 106 provides information in a visual and understandable format, including accounts and payment information, into visual representations that are easy to comprehend.

In some non-limiting embodiments or aspects, data visualization system 106 acts as a vital tool for comprehending and leveraging data across the supply chain. Data visualization system 106 provides seamless communication with other enterprise systems, transforms complex information into easily understandable visuals, provides actionable inferences based on processed data, and contributes to optimized value analysis and item management. Through these functions, data visualization system 106 enhances the overall functioning of the SCM system and its ability to handle contract management effectively.

Data store 108 provides a repository for various types of data that are relevant to procurement prediction engine 102. This may include historical transaction data, account information, supplier details, past exceptions, and other relevant datasets. The data may be stored in an organized manner, making it easily retrievable when needed for analysis, modeling, and prediction.

In some non-limiting embodiments or aspects, procurement prediction engine 102 interacts with data store 108 to access (or store) the necessary information for its predictive analysis. It may retrieve data from the database to build and train prediction models. The data store acts as a central hub where different sources of data are integrated, enabling the procurement prediction engine to have a comprehensive view of the healthcare supply chain's historical activities and patterns.

In some non-limiting embodiments or aspects, machine learning models operating within procurement prediction engine 102 may include a significant storage amount of training data to make accurate predictions. Data store 108 maintains or provides this data for model training purposes. Procurement prediction engine 102 may obtain or receive relevant data from the database to train and refine its prediction algorithms.

In some non-limiting embodiments or aspects, computing environment 100 includes a healthcare supply chain, such that new supply chain data is constantly generated. Data store 108 provides real-time or periodic updates to ensure that the prediction models are up-to-date and reflective of the latest trends and activities.

In some non-limiting embodiments or aspects, data store 108 performs data cleansing and preprocessing. For example, before using the data, procurement prediction engine 102 may preprocess the data (e.g., automatically preprocess, etc.), cleanse it, remove inconsistencies, remove errors, or remove irrelevant information. This preprocessing may include tasks such as data normalization, handling missing values, and data transformation. Data store 108 may potentially include features to assist with these tasks.

In some non-limiting embodiments or aspects, data store 108 provides data security and access control. For example, data stored in data store 108 may be sensitive and valuable. data store 108 determines robust security measures to protect the data, including user authentication, access controls, encryption of data at rest, and audit trails to track who accessed the data and when, and/or the like.

In some non-limiting embodiments or aspects, data store 108 provides scalability and performance: For example, data store 108, as the amount of data grows over time, provides scalability and performance. Data store 108 may be configured to efficiently handle large volumes of data and provide rapid access for analysis and predictions.

In some non-limiting embodiments or aspects, data store 108 provides retrospective analysis, reporting, and generating inferences into past sourcing of contracts and supply chain activities to identify in combination with trends, areas for improvement, and potential strategies to source future contracts.

In some non-limiting embodiments or aspects, user computing device 112 may be a personal computing device (e.g., desktop computer, laptop computer) or mobile computing device (e.g., smartphone, tablet). In addition, user computing device 112 may be linked to and/or used by an enterprise user (e.g., who may be an employee of a healthcare institution operating procurement prediction engine 102). For example, user computing device 112 may be used by an enterprise associate who manually processes and/or reviews sourcing activities or items. Additionally, user computing device 112 includes functions for processing and review of sourcing activities, as well as configuration and monitoring of procurement prediction engine 102 and other enterprise computing devices.

Supplier sales automation system 120 also may be a personal computing device (e.g., desktop computer, laptop computer) or mobile computing device (e.g., smartphone, tablet). In addition, supplier sales automation system 120 may be linked to and/or used by an enterprise user (who may, e.g., be an employee of a healthcare institution operating procurement prediction engine 102). For example, supplier sales automation system 120 may be used by a network administrator or backend enterprise user who monitors and/or configures procurement prediction engine 102 and/or other enterprise computing devices.

External computing system 118 may include one or more computing devices and/or other computer components (e.g., processors, memories, communication interfaces). In addition, and as illustrated in greater detail below, external computing system 118 may be linked to and/or used by an external organization (e.g., an organization different from a healthcare institution operating procurement prediction engine 102). For example, external computing system 118 may be used by a third-party healthcare institution in presenting and/or otherwise submitting one or more account items to procurement prediction engine 102 and/or a healthcare institution operating procurement prediction engine 102.

Computing environment 100 also may include one or more networks, which may interconnect one or more of procurement prediction engine 102, resource planning system 104, and data visualization system 106, data store 108, user computing device 112, external computing system 118, and supplier sales automation system 120. For example, computing environment 100 may include private network 114 (which may interconnect with a procurement prediction engine 102 and public network 116 (which may interconnect supplier sales automation 120 and/or external computing systems 118 with private network 114, one or more other systems, public networks, sub-networks, and/or the like).

In some non-limiting embodiments or aspects, user computing device 112 may monitor and configure the procurement inference engine and other enterprise computing devices, and also facilitates the monitoring and configuration of the procurement inference engine and other enterprise computing devices. This active oversight ensures that these systems operate optimally, align with business objectives, and respond effectively to changing conditions.

In some non-limiting embodiments or aspects, resource planning system 104, data visualization system 106, user computing device 112, external computing system 118, supplier sales automation system 120, and/or the other systems included in computing environment 100 may be any type of computing device capable of receiving a user interface, receiving input via the user interface, and communicating the received input to one or more other computing devices. For example, resource planning system 104, data visualization system 106, user computing device 112, external computing system 118, supplier sales automation system 120, and/or the other systems included in computing environment 100 may, in some examples, be server computers, desktop computers, laptop computers, tablet computers, smart phones, or the like, that may include one or more processors, memories, communication interfaces, storage devices, and/or other components. As noted above, and as illustrated in greater detail below, any and/or all of procurement prediction engine 102, resource planning system 104, data visualization system 106, user computing device 112, external computing system 118 and supplier sales automation system 120, may, in some examples, be special-purpose computing devices configured to perform specific functions.

In some non-limiting embodiments or aspects, external computing system 118 provides an electronic interface and connector between external entities and the internal systems of the supply chain framework.

In some non-limiting embodiments or aspects, external computing system 118 submits account items and relevant data for contract management processing. For example, external computing system 118 allows external organizations (e.g., one or more third-party healthcare institutions, suppliers, etc.) to submit account items and associated data to procurement prediction engine 102. This function facilitates the inclusion of external data in the predictive analysis and in the contract management handling processes.

In some non-limiting embodiments or aspects, external computing system 118 facilitates communication and integration with the procurement prediction ending 102. For example, external computing system 118 serves as a bridge for communication and integration between the external elements and procurement prediction engine 102, so that data and information flow efficiently between elements of the SCM system.

In some non-limiting embodiments or aspects, external computing system 118 is configured to interact with the resource planning system and the data visualization system. For example, external computing system 118 is capable of interacting with both the resource planning system and the data visualization system. This interaction includes a secure connection of the external entity to engage with and access relevant information and inferences from one or more systems.

In some non-limiting embodiments or aspects, external computing system 118 supports the transmission of supply information within a supplier system. For example, external computing system 118 obtains supply-related information within a supplier's system, for accurate and up-to-date records and data within the supplier's domain.

External computing system 118 provides a communication hub and a liaison between external entities and the internal SCM systems. External computing system 118 receives external data, integrates with the procurement prediction platform, interacts with the resource planning and the data visualization system 106, and facilitates the exchange of supply-related information within a supplier's system. This integration includes the flow of information, collaboration, and overall efficiency within the SCM ecosystem.

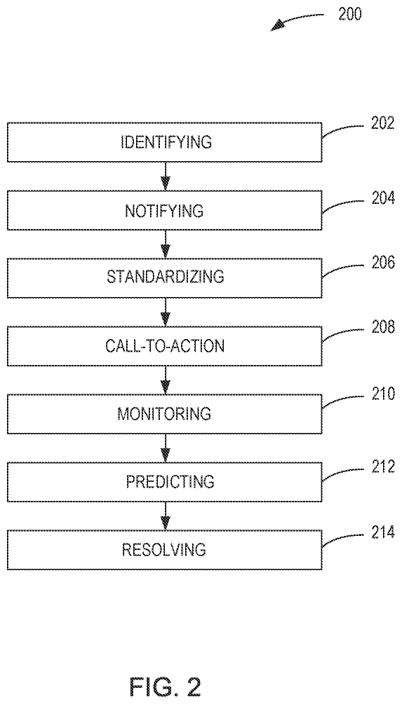

In some non-limiting embodiments or aspects, procurement prediction engine 102 includes one or more processors, memories, and communication interfaces, as described in further detail with reference to FIG. 2. A data bus interconnects the processor, memory, and communication interface. The memory contains program modules and processing engines with instructions that, when executed by the processor, enable procurement prediction engine 102 to perform functions as described herein. It also stores databases that maintain information used by these program modules, processing engines, and the processor. In certain examples, these program modules, processing engines, and databases are stored in different memory units of procurement prediction engine 102 or different computing devices that make up procurement prediction engine 102. For example, the memory may also include a contract management processing module, a contract management processing database, and a contract management item learning engine.

Procurement prediction engine 102 may perform improved sourcing processing using cognitive automation tools, as discussed in greater detail below. data store 108 (e.g., exception processing database, exception database programming, etc.) may store sourcing prediction data or may generate sourcing prediction information using programming in procurement prediction engine 102, such as contract management learning in procurement prediction engine 102 to perform one or more cognitive automation and/or machine learning function and/or service, as illustrated in greater detail below.

In some non-limiting embodiments or aspects, procurement prediction engine 102 utilizes machine learning techniques (e.g., supervised and unsupervised learning, e.g., predictive models for identifying supply chain anomalies, sentiment analysis of supplier communications, etc.) for generating models. Procurement prediction engine 102 analyzes training data to create prediction models, classification models, segmentation models, etc. (e.g., predicting demand for specific medical supplies, classifying suppliers based on performance levels, etc.). Procurement prediction engine 102 employs new models and advancements in computational power for practical applications (e.g., incorporating state-of-the-art deep learning models for improved accuracy, leveraging faster GPUs for quicker predictions, etc.). Procurement prediction engine 102 supports inference engines for executing model algorithms and generating inference outputs (e.g., suggesting optimal reorder quantities, highlighting potential fraudulent activities, etc.). Procurement prediction engine 102 generates and provides mission-critical healthcare information using machine learning (e.g., forecasting supply chain disruptions, predicting patient demand patterns, etc.). Procurement prediction engine 102 automates processes and actions such as interpreting communications and predicting responses (e.g., automatically flagging discrepancies in transaction data, predicting supplier responses to potential delays, etc.).

In some non-limiting embodiments or aspects, procurement prediction engine 102 engages in supervised learning by approximating the mapping function from known input variables to output variables for predictions (e.g., predicting future order quantities based on historical data, estimating lead times for suppliers, etc.). Procurement prediction engine 102 addresses regression problems, solving for real-value output variables (e.g., dollars or items, e.g., forecasting future costs based on historical spending patterns, predicting the price of medical supplies, etc.). Procurement prediction engine 102 tackles classification problems, as an example, solving for categorical output variables (e.g., pending/closed, compliant/non-compliant, e.g., categorizing transactions as urgent or non-urgent, classifying suppliers into high-risk or low-risk categories, etc.).

In some non-limiting embodiments or aspects, procurement prediction engine 102 also utilizes unsupervised learning to model the underlying structure or distribution in the data (e.g., identifying patterns in supplier behaviors, clustering similar types of orders, etc.). Procurement prediction engine 102 addresses clustering problems, discovering inherent groupings in a dataset (e.g., grouping similar suppliers based on performance metrics, clustering orders based on delivery locations, etc.). Procurement prediction engine 102 deals with the association rule learning problem, discovering rules that describe large portions of data (e.g., identifying purchasing patterns based on historical transaction data, uncovering associations between suppliers and product categories, etc.).

In addition to identifying patterns based on transaction history or uncovering associations between suppliers and product categories, procurement prediction engine 102 may provide customizable filters within procurement prediction engine 102. For example, users applying their own criteria to filter data before using procurement prediction engine 102, including operation of a user interface for implementing such filters. In such an example, recent activities are filtered out for a specific period of time before being included in the analysis (e.g., 10 days, a month, etc.).

In some non-limiting embodiments or aspects, procurement prediction engine 102 is trained for machine learning (e.g., one or more models are trained, etc.) via various techniques and algorithms (e.g., utilizing decision trees to predict contract fulfillment outcomes, employing neural networks to forecast demand fluctuations within the C2C process, etc.). Procurement prediction engine 102 analyzes training data to generate models for problem-solving in diverse variations (e.g., creating different prediction models for supplier behavior in contract management, adjusting models based on different product categories relevant to contract management, etc.). Procurement prediction engine 102 employs cognitive automation tools to enhance supplier processing within the contract management framework (e.g., automating communication with suppliers to resolve automatically, routing contract-related matters to appropriate teams based on predictions within the contract management process, etc.). Procurement prediction engine 102 predicts actions, responses, and interprets operating parameters correlated with critical parameters within the C2C context (e.g., predicting when to expedite contract orders based on changing demand patterns in contract management, interpreting supplier responses to contract management contract delays, etc.). Procurement prediction engine 102 increases efficiencies in healthcare records, data storage, and item management within the contract management process (e.g., optimizing inventory levels to minimize excess stock and shortages specifically related to contract management contracts, efficiently managing supply lifecycle within contract management, etc.). Procurement prediction engine 102 enhances workflow and targeted communications for efficiency and accuracy within the contract management process (e.g., automating handling of contract management pipelines, sending real-time alerts to relevant stakeholders based on predictions within contract management, etc.).

In some non-limiting embodiments or aspects, private network 114 and public network 116 may include one or more wired and/or wireless networks. For example, private network 114 and public network 116 may include a cellular network (e.g., a long-term evolution (LTE) network, a third generation (3G) network, a fourth generation (4G) network, a code division multiple access (CDMA) network, and/or the like), a public land mobile network (PLMN), a local area network (LAN), a wide area network (WAN), a metropolitan area network (MAN), a telephone network (e.g., the public switched telephone network (PSTN)), a private network (e.g., a private network associated with a transaction service provider), an ad hoc network, an intranet, the Internet, a fiber optic-based network, a cloud computing network, and/or the like, and/or a combination of these or other types of networks.

The number and arrangement of systems, devices, and/or networks shown in FIG. 1 are provided as an example. There may be additional systems, devices, and/or networks; fewer systems, devices, and/or networks; different systems, devices, and/or networks; and/or differently arranged systems, devices, and/or networks than those shown in FIG. 1. Furthermore, two or more systems or devices shown in FIG. 1 may be implemented within a single system or device, or a single system or device shown in FIG. 1 may be implemented as multiple, distributed systems or devices. Additionally or alternatively, a set of systems (e.g., one or more systems) or a set of devices (e.g., one or more devices) of computing environment 100 may perform one or more functions described as being performed by another set of systems or another set of devices of computing environment 100.

Referring now to FIG. 2, FIG. 2 is a diagram of a non-limiting embodiment of a workflow 200 for automating contract management. In some non-limiting embodiments, one or more of the steps of workflow 200 are performed (e.g., completely, partially, etc.) by procurement prediction engine 102. For example, one or more steps of workflow 200 are performed by procurement prediction engine 102 (e.g., one or more devices of procurement prediction engine 102), resource planning system 104 (e.g., one or more devices of resource planning system 104, etc.), data visualization system 106 (e.g., one or more devices of data visualization system 106), data store 108 (e.g., one or more tables, one or more linked tables, one or more linked databases, etc.), internal sales automation 110, user computing device 112 (e.g., one or more devices of user computing device 112), external computing system 118 (e.g., one or more devices of external computing system 118), and a supplier sales automation system 120 (e.g., one or more devices of supplier sales automation 120).

As shown in FIG. 2, at step 202, workflow 200 includes identifying. In some non-limiting embodiments or aspects, procurement prediction engine 102 identifies issues. For example, during contract management, issue identification includes the recognition of potential challenges, discrepancies, or delays within the contract lifecycle.

In some non-limiting embodiments or aspects, procurement prediction engine 102 provides cognitive automation tools and machine learning algorithms for reviewing contract-related data to pinpoint patterns, anomalies, or deviations from standard processes that may signal potential issues.

In some non-limiting embodiments or aspects, resource planning system 104 monitors and manages contracts within the broader organizational context and may also flag issues related to resource allocation, financial planning, or other factors impacting contract execution.

In some non-limiting embodiments or aspects, data visualization system 106 provides a visual representation of contract-related data, aiding issue identification by presenting key performance indicators, trends, or areas of concern. In other examples, data store 108 provides a repository for contract-related data, actively supporting issue identification by providing historical transaction data and relevant information. In some examples where contracts are linked to sales activities, internal sales automation 110 may provide issue identification by highlighting any discrepancies or challenges in the sales-related aspects of contract execution.

As shown in FIG. 2, at step 204, workflow 200 includes notification. In some non-limiting embodiments or aspects, procurement prediction engine 102 provides automated notification. For example, procurement prediction engine 102 informs relevant stakeholders about identified issues, potential risks, or necessary actions related to contracts. For example, procurement prediction engine 102 automates notifications with cognitive automation tools by automatically generating notifications based on contract data. Procurement prediction engine 102 may utilize machine learning algorithms to predict issues, deviations, or trends that may impact contract performance.

In some non-limiting embodiments or aspects, resource planning system 104 collaborates with procurement prediction engine 102 by providing insights into resource allocation, financial considerations, and other factors that contribute to the context of the automated notifications. In such an example, data visualization system 106 presents visually accessible representations of the contract data to aid stakeholders in understanding the notifications. User computing devices 112 may be used for receipt and acknowledgment of automated notifications, enabling human oversight and decision-making. Internal sales automation 110 may provide further automation of notifications related to sales aspects tied to contracts. For example, procurement prediction engine 102 provides automated notification to ensure that relevant information is communicated to stakeholders through notifications automatically allowing for timely response in the contract management process.

FIG. 2, at step 206, workflow 200 includes standardizing. For example, procurement prediction engine 102 standardizes by activating processes and procedures for resolving identified issues or discrepancies in contracts. Standardizing may refer to the process of establishing and adhering to a set of guidelines, specifications, or protocols that ensure uniformity, consistency, and interoperability within a system or across multiple systems. Standardization includes creating and executing a common framework (e.g., a set of rules, etc.) that enables different components, systems, or processes to work together seamlessly based on conventions, formats, and interfaces that allow for compatibility and integration. In addition, procurement prediction engine 102 standardizes by facilitating communication and interaction between activities, actions, communications, and/or the like. In the context of contract management, standardizing processes ensures that workflows, data formats, communication protocols, and other aspects follow a consistent and predefined structure, promoting efficiency, reliability, and ease of maintenance. Procurement prediction engine 102 may initiate (e.g., cause to execute, etc.) one or more automated workflows, to provide standardized, and consistent resolution of issues within the contract management process.

In some non-limiting embodiments or aspects, procurement prediction engine 102 generates or executes automation tools to trigger automated workflows based on one or more predictions formed from contract-related data.

In some non-limiting embodiments or aspects, resource planning system 104 provides information so that procurement prediction engine 102 may provide inferences into resource allocation, financial implications, and other relevant parameters. Procurement prediction engine 102 may align these aspects with the automated workflows. Data visualization system 106 offers a visual representation of the workflows and associated issues, for stakeholders reviewing and monitoring the standardized processes. User computing devices 112 provide input and decision-making to procurement prediction engine 102. Internal sales automation 110 may contribute by automating workflows related to sales components integrated with contracts.

As shown in FIG. 2, at step 208, workflow 200 includes call-to-action. For example, procurement prediction engine 102 determines or generates a call-to-action. In some non-limiting embodiments or aspects, when determining the call-to-action, procurement prediction engine 102 generates notifications that are directed to the appropriate stakeholders (e.g., the correct individuals to involve in in the response or resolution process, etc.). This may include contract managers, legal teams, procurement officers, other relevant parties, and/or the like. For example, when an issue or exception is identified in the contract management process, automated notifications are sent to specific individuals or teams. For example, individuals who have the expertise or authority to address and resolve the issue. During this step in the automatic contract management activation processing, systems such as procurement prediction engine 102, resource planning system 104, and data store 108 may be active. For example, procurement prediction engine 102 provides a targeted and efficient response to the problem, such as, identifying the issue. In some examples, resource planning system 106 may determine the appropriate stakeholders based on predefined rules. In some examples, data store 108 may obtain, store, access, or provide information associated with the contracts and stakeholders.

As shown in FIG. 2, at step 210, workflow 200 includes monitoring. For example, procurement prediction engine 102 provides real-time monitoring of processes associated with communications, actions, and resolutions. In some non-limiting embodiments or aspects, procurement prediction engine 102 coordinates a response.

In some non-limiting embodiments or aspects, procurement prediction engine 102 may track the progress of an issue resolution, view status updates, and receive alerts for actions. For example, procurement prediction engine 102 tracks one or more actions required for stakeholders. In such an example, procurement prediction engine 102 may provide continuous and immediate oversight of a communication or resolution activity. Procurement prediction engine 102 may provide directly or through other systems, an ability for stakeholders involved in a contract management process to actively monitor the advancement of issue resolution.

In some non-limiting embodiments or aspects, procurement prediction engine 102 generates and communicates real-time updates on the status of ongoing processes, provides timely alerts or notifications prompting stakeholders to take necessary actions, and/or the like. In some examples, procurement prediction engine 102 may integrate with the data visualization system 106 or an email automation system to provide real-time status updates, visual representations of the resolution process, alerts to stakeholders, and/or the like. Monitoring in real-time provides information that stakeholders may use to interact with the system, view information, respond to alerts, collaborate in the contract management lifecycle, and/or the like.

As shown in FIG. 2, at step 212, workflow 200 includes predicting. In some non-limiting embodiments or aspects, procurement prediction engine 102 predicts issues, actions, responses, resolutions, and/or the like. For example, procurement prediction engine 102 conducts predictive trend analysis to predict potential communication bottlenecks, issues, actions, responses, resolutions, and/or the like. In such an example, procurement prediction engine 102 anticipates actions, thereby reducing the likelihood of delays.

In some non-limiting embodiments or aspects, procurement prediction engine 102 executes advanced analytical techniques and machine learning algorithms as discussed herein to analyze historical data and identify patterns. Procurement prediction engine 102 may predict potential communication challenges or obstacles in the contract management process. Procurement prediction engine 102 or another engine as described below may then conduct proactive trend analysis, to anticipate issues before they occur. This predictive capability includes anticipatory actions, mitigation actions, removal of potential delays in the contract management workflow, and/or the like. Data store 108 may provide the historical data necessary for trend analysis.

As shown in FIG. 2, at step 214 workflow 200 includes resolution. For example, procurement prediction engine 102 initiates actions for resolution. In some examples, as the activities for contract management advance, stakeholders actively contribute to the resolution of the identified issue. Procurement prediction engine 102 monitors the completion of these workflows. Upon successful resolution, procurement prediction engine 102 initiates closure procedures, signifying the conclusion of the communication and resolution cycle. In some examples, resource planning system 104 likely plays a role in coordinating and managing the progress of workflows, while data store 108 may store relevant data and status updates. Data visualization system 106 may provide visual representations of the resolution status, or provide an interface through which stakeholders participate in the resolution process and receive closure notifications.

In some non-limiting embodiments or aspects, procurement prediction engine 102, resource planning system 104, data visualization system 106, or internal sales automation 110 systematically capture and record detailed information about each communication or activity related to issue resolution. Procurement prediction engine 102 may activate resolution based on this information, including an activation based on timestamps for when communications occurred, decisions made during the process, the actions taken to address issues, and/or the like. The automated documentation feature ensures that there is a thorough and traceable record of the communication history throughout the resolution process. In some examples, data visualization system 106 presents a visualization of the documented information in a comprehensible format. In some examples, data store 108 stores and/or manages the comprehensive records and provides stakeholders access for transparency and accountability purposes.

In some non-limiting embodiments or aspects, procurement prediction engine 102 evolves processes to account for changing regulatory standards and industry trends. This adaptability ensures that the processes for streamlining communication in contract management remains adapted in accordance with current regulations and industry best practices. Procurement prediction engine 102 may provide insights into current regulatory landscape and industry trends, to adjust its configurations accordingly. Additionally, data store 108 plays a role in storing and retrieving relevant information about regulatory requirements, and/or provides interface for users to configure and update the system settings based on changing standards.

In some non-limiting embodiments or aspects, procurement prediction engine 102 may include a feedback loop for continuous improvement and optimization of the automated activities, actions, responses, and resolutions. For example, procurement prediction engine 102 may make updates based on stakeholders' feedback regarding effectiveness of the communication and resolution processes. This feedback loop is integral for ongoing enhancement and optimization of the automated communication workflows. In some non-limiting embodiments or aspects, feedback information provides information for continuous improvement and adjustment of the automated communication workflows in the contract management system.

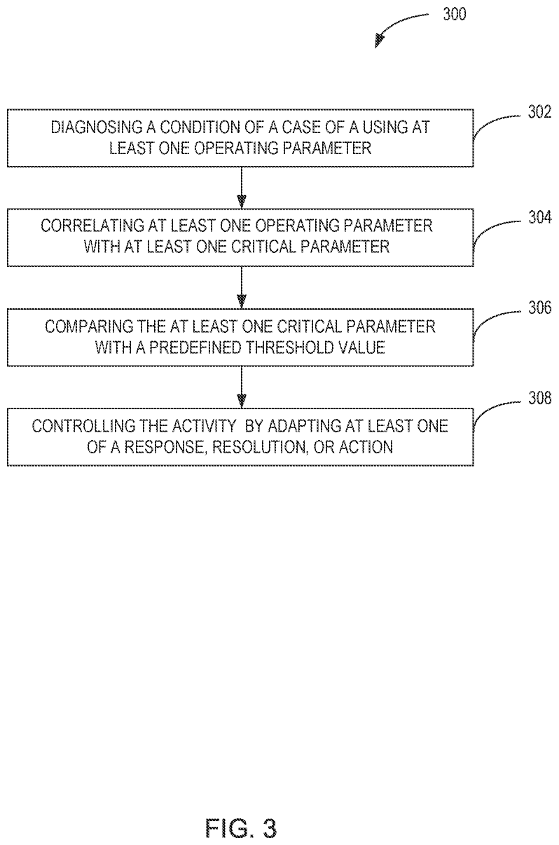

Referring now to FIG. 3, FIG. 3 is a step diagram of a non-limiting embodiment of a process 300 for automatic contract management activation. In some non-limiting embodiments, one or more of the steps of process 300 are performed (e.g., completely, partially, etc.) by computing environment 100 and may include one or more computer systems. For example, computing environment 100 may include procurement prediction engine 102 (e.g., one or more devices of procurement prediction engine 102, one or more processors, one or more perception nodes, one or more inference engines, and/or the like of procurement prediction engine 102), resource planning system 104 (e.g., one or more processors of resource planning system 104, etc.), data visualization system 106 (e.g., one or more processors of data visualization system 106), user computing device 112 (e.g., one or more devices of user computing device 112, etc.), external computing system 118 (e.g., one or more devices of external computing system 118, etc.) and supplier sales automation 120 (e.g., one or more devices of supplier sales automation 120, one or more processors, or procurement prediction engine 102, etc.).

As shown in FIG. 3, at step 302, process 300 includes diagnosing a condition of a case of a resource planning system using at least one operating parameter. For example, procurement prediction engine 402 diagnoses a condition of a case of a resource planning system using at least one operating parameter.

In some non-limiting embodiments or aspects, the one or more operating parameters are formed from at least one specified field and are correlated to at least one critical parameter including at least one reason for a contract renewal, contract maintenance, or a requisition placement. For example, procurement prediction engine 102 includes one or more perception nodes for executing a correlation process between operating parameters and critical parameters. Procurement prediction engine 102 may comprise neural networks (e.g., one or more neural networks of one or more perception nodes, etc.) trained to predict activities aligned with critical parameters. In some examples, they are responsible for analyzing the specified fields and forming operating parameters. Based on training, procurement prediction engine 102 is able to discern patterns, trends, and anomalies within the operational data.

In some non-limiting embodiments or aspects, procurement prediction engine 102 perception nodes within the procurement prediction engine process the operational data from specified fields within a contract management system. Procurement prediction engine 102 may extract relevant information and generate operating parameters.

Procurement prediction engine 102 correlates these operating parameters with critical parameters associated with contract management activities. Activities for contract renewal, contract maintenance, requisition placement, and/or the like may be associated with critical parameters. Procurement prediction engine 102 provides correlation by inferring relationships and/or dependencies between the operational indicators and the reasons for specific actions.

Procurement prediction engine 102 predicts activities that align (e.g., match, or correlate within a threshold, etc.) with critical parameters. For example, when procurement prediction engine 102 identifies certain operational parameters associated with successful contract performance, the perception engine can predict a recommendation for contract renewal.

In some non-limiting embodiments or aspects, based on the results of the correlation and prediction, procurement prediction engine 102 adapts the contract management system. The adaptation may include adjusting responses, communication strategies, specific actions, and/or the like, within the contract management workflow. For example, when the prediction suggests a high likelihood of contract renewal, the system may adapt by initiating the renewal process.

Procurement prediction engine 102, through correlation and prediction, enhances the decision-making capabilities of the contract management system by initiating one or more actions that are aligned with the identified critical parameters and operational realities.

In some non-limiting embodiments or aspects, procurement prediction engine 102 (e.g., the one or more nodes and the neural network, etc.) are trained to make the correlation and predictions. In some non-limiting embodiments or aspects, procurement prediction engine 102 determines the at least one operating parameter; correlates the operating parameter with a value for the at least one critical parameter; compares the determined value of the at least one critical parameter with at least one critical parameter diagnosed during training of the one or more perception nodes and the neural network. In some non-limiting embodiments or aspects, when determining non-conformance or too large a deviation, procurement prediction engine 102 adapts the correlation module and the neural network, and repeats the steps.

In some non-limiting embodiments or aspects, procurement prediction engine 102, in response to determining conformance or a deviation below a predefined limit value, terminates the training and transmits the trained neural network to purchase order rectifier systems of the same type. The procurement prediction engine utilizes two machine learning (ML) models, referred to as the first ML model and the second ML model. Each model generates inference results based on input data and the patterns it has learned.

In some non-limiting embodiments or aspects, procurement prediction engine 102 determines conformation or deviation based at least in part on a first plurality of inference results and a second plurality of inference results. Procurement prediction engine 102 determines the inference results from both models, resulting in a determination of two responses—a first response corresponding to the first ML model and a second response corresponding to the second ML model. Each response signifies a different recommended action or decision based on the inferences generated by the respective ML model.

Procurement prediction engine 102 determines a first response corresponding to the first ML model and a second response corresponding to the second ML model. The responses are evaluated to determine the precedence between the two ML models. When the first response indicates that the first ML model has higher precedence than the second ML model, it suggests that the inferences from the first model should be given priority. A model selector mechanism is then activated based on the determined precedence. This model selector is responsible for choosing between the first ML model and the second ML model for generating inferences in response to future inference requests.

In some non-limiting embodiments or aspects, each response indicates a different response by the inferences generated by the corresponding ML model, wherein the first response is selected when it indicates that the first ML model is of higher precedence than the second ML model. The model selector dynamically distributes inference requests, allocating more requests to the ML model with higher precedence (e.g., the one indicated by the first response) than what was previously distributed. This distribution process is part of an adaptive strategy, ensuring that the model with higher precedence handles a greater share of inference requests in line with its perceived reliability or accuracy. For example, procurement prediction engine 102 provides a model selector to cause the model selector to select between the first ML model and the second ML model for generating inferences for inference requests according to a call to action. In some examples, the model selector distributes inference requests that are to be provided to the first ML model than as indicated by a previous distribution with a higher precedence to the second ML model.

In some non-limiting embodiments or aspects, procurement prediction engine 102 includes further instructions, which when executed cause the procurement inference engine to perform an analysis of pricing review, product comparison, agreement review, wherein pricing review comprises a statistical analysis to model preferences for a product or service at various price points. For example, procurement prediction engine 102 determines a pricing review based on a review (e.g., examination, etc.) of pricing data related to products or services.

As an example, procurement prediction engine 102 utilizes statistical methods to analyze the pricing data. This statistical analysis aims to model preferences for a product or service across different price points. The focus of the analysis may provide inferences for understanding how preferences for a particular product or service vary concerning a price. Procurement prediction engine 102 may identify patterns or trends in user or stakeholder preferences in relation to pricing. In another example, procurement prediction engine 102 generates product comparisons. For example, procurement prediction engine 102 assesses and compares various products based on specified criteria for decision-making by providing relative merits and features of different products. Procurement prediction engine 102 may perform an agreement review (e.g., an examination of contractual agreements, terms, and conditions related to procurement) of the terms and conditions of agreements for compliance, identify risks, and optimize the procurement process to analyze pricing data statistically, compare products, and review agreements.

In some examples, procurement prediction engine 102 provides capability to diagnose a contract renewal in a first ML model, diagnose a contract maintenance in a second ML model, and diagnose a requisition placement in a third ML model. Three ML models are configured, each specialized in diagnosing a specific aspect of the contract management lifecycle: A first ML model focuses on diagnosing contract renewals or diagnoses the need for a contract renewal. A second ML model specializes in diagnosing contract maintenance. A third ML model is configured for diagnosing requisition placements.

The models determine an appropriate action to take based on inference results. These inference results are associated with specific aspects such as the initiator, expiration date, owner, and contract information. The response type is linked to various contract scenarios, including emergency preparedness, placement, consignment, service, construction, evaluation, group purchasing organization (GPO), or local pricing.

In some non-limiting embodiments or aspects, first ML model diagnoses an action to take for a contract renewal, wherein one or more inference results are associated with at least one of an initiator, an expiration date, an owner, contract information for a response type associated with at least one of emergency preparedness, a placement, a consignment, a service, a construction, an evaluation, a GPO, or a local pricing.

The second ML model diagnoses an action to take for a contract maintenance by determining a critical factor associated with a reason for the contract maintenance from at least one of assigning to owner, cancelation, update or amend current contract, price change, performance notification. The second ML model diagnoses the need for contract maintenance. The second ML model identifies a critical factor associated with the reason for contract maintenance. These critical factors may include assigning an owner, cancellation, current contract status, price changes, or performance notifications.

In some non-limiting embodiments or aspects, diagnose, by the third ML model, an assignment of a requisition placement to a commodity. The third ML model diagnoses the assignment of a requisition placement to a commodity. Procurement prediction engine 102, with different ML models to diagnose specific aspects of contract management, determines appropriate actions based on the diagnosis, and associates inference results with relevant details for effective decision-making in areas like contract renewals, maintenance, and requisition placements.

As shown in FIG. 3, at step 304, process 300 includes correlating at least one operating parameter with at least one critical parameter of the resource planning system. For example, procurement prediction engine 102 correlates at least one operating parameter with at least one critical parameter of the resource planning system. For example, procurement prediction engine 102 correlates, with one or more perception nodes, at least one operating parameter with at least one critical parameter of the resource planning system, In such an example, the one or more perception nodes comprise a neural network trained to correlate an activity with the at least one critical parameter.

In some non-limiting embodiments or aspects, procurement prediction engine 102 determines at least one operating parameter of the contract management system. These operating parameters might relate to the ongoing processes and activities within the system. The neural network is trained to predict activities aligned with critical parameters. Critical parameters may include aspects crucial for the effective functioning of the contract management system, such as resolution time, accuracy, workflow efficiency, and/or the like.

In some non-limiting embodiments or aspects, procurement prediction engine 102 generates a prediction. As an example, the prediction (e.g. structured information, a message, vector, etc.) for a forecast or to anticipate activities aligned with critical parameters. The prediction may be based on the correlation of operating parameters with critical parameters. Essentially, the system predicts what activities or conditions are likely to occur based on its training.

In summary, comparison results are the outcomes of assessing the current state of the contract management system against predefined criteria. Predictions, on the other hand, involve anticipating future activities or conditions based on the correlation of parameters. While they both contribute to the overall decision-making process, they are distinct steps in the sequence of actions described in the process.

Predictions, generated by the neural network based on the correlation of operating parameters, anticipate future activities aligned with critical parameters. These predictions are then used to guide actions, responses, and resolutions in the contract management activities. For example, when a prediction indicates a potential issue in the future, the system can proactively initiate actions or responses to prevent or address the predicted problem.

In this way, the comparison results inform adaptations to the system's current state, while the prediction results guide proactive responses and resolutions to potential future scenarios in the context of contract management activities.

Still referring to FIG. 3, at step 306, process 300 includes comparing the at least one critical parameter with a predefined threshold value. For example, procurement prediction engine 102 compares the at least one critical parameter with a predefined item threshold value. For example, comparing critical parameters with threshold values, the critical parameters identified are compared with predefined threshold values. Procurement prediction engine 102 determines whether the contract management system may be operating within acceptable limits or when there are deviations that require attention.

In some non-limiting embodiments or aspects, comparison results refer to the outcome of the step where critical parameters of the contract management system are compared with predefined threshold values. The comparison results assess whether the system's performance or conditions meet acceptable standards or when there are deviations that require attention. In some examples, the result of this comparison informs subsequent actions to be taken.

In some non-limiting embodiments or aspects, procurement prediction engine 102 obtains results by comparing critical parameters with predefined threshold values, procurement prediction engine 102 may determine the current state of the contract management system. When discrepancies or issues are identified, procurement prediction engine 102 may adapt the contract management system. For example, when a critical parameter falls below the predefined threshold, the system might need to adjust its settings or operations to address the deviation and maintain optimal performance.

In some non-limiting embodiments or aspects, the critical parameter (e.g., the at least one critical parameter, etc.) is used in both steps.

In some non-limiting embodiments or aspects, the critical parameter plays a dual role in the system. In the comparison step, the critical parameter may be assessed against a predefined threshold value. This comparison helps determine whether the current state of the system aligns with the expected or desired state. When the critical parameter deviates significantly from the predefined threshold, it triggers the need for adaptation in the contract management system.

In the prediction step, the same critical parameter may be used by one or more perception nodes in a neural network to foresee future activities. The neural network may be trained to predict activities aligned with the critical parameter, providing a proactive understanding of potential issues or trends that might arise.

The implication may be that the critical parameter may be a key metric for both assessing the current state and predicting future activities. It serves as a crucial factor for decision-making in both adapting the system based on current conditions and proactively responding to anticipated events. This dual usage underscores the importance of the chosen critical parameter in the overall functioning and optimization of the contract management system.

As shown in FIG. 3, at step 308, process 300 includes controlling the activity of a contract management by adapting at least one of a response, communication, or action of a contract management system. For example, procurement prediction engine 102 controls the activity of a contract management by adapting at least one of a response, communication, or action of a contract management system. In some non-limiting embodiments or aspects, procurement prediction engine 102 controls the activity of a contract management activity, based on the prediction of the activity aligned with the at least one critical parameter. For example, procurement prediction engine 102 generates at least one of a response, communication, or action of a contract management system. In some non-limiting embodiments or aspects, procurement prediction engine 102 adapts the contract management system by automatically adjusting settings or operations to eliminate the at least one deviation to maintain optimal performance.

In some non-limiting embodiments or aspects, the at least one critical parameter plays a dual role, used in both steps of the critical parameter for comparison and prediction.

In the comparison step, the critical parameter may be assessed against a predefined threshold value. This comparison helps determine whether the current state of the system aligns with the expected or desired state. When the critical parameter deviates significantly from the predefined threshold, it may provide triggers for adaptation in the contract management system.

In the prediction step, the same critical parameter may be used by one or more perception nodes in a neural network to forecast future activities. Procurement prediction engine 102 may be trained to predict activities aligned with the critical parameter, providing a proactive understanding of potential issues or trends that might arise.

In some non-limiting embodiments or aspects, critical parameter may be a key metric for both assessing the current state and predicting future activities. It serves as a crucial factor for decision-making in both adapting the system based on current conditions and proactively responding to anticipated events. This dual usage underscores the importance of the chosen critical parameter in the overall functioning and optimization of the contract management system.

In some non-limiting embodiments or aspects, procurement prediction engine 102 controlling contract management activities based on the comparison results, adaptive actions are taken to control the activities of the contract management system. Procurement prediction engine 102 may modify responses, enhancing communication protocols, or adjusting actions to address any issues or optimize performance. Procurement prediction engine 102 refers to the outcome of the step where critical parameters of the contract management system are compared with predefined threshold values. The purpose of this step may be to assess whether the system's performance or conditions meet acceptable standards or when there are deviations that need attention. The result of this comparison informs subsequent actions to be taken.

When procurement prediction engine 102 adapts the contract management system, procurement prediction engine 102 may automatically adjust settings or operations to eliminate the at least one deviation to maintain optimal performance.

In some non-limiting embodiments or aspects, the deviation may be a discrepancy in a contract where the terms outlined in the contract are not being adhered to or are at risk of not being fulfilled. For example, a non-compliance where a party involved in the contract may not be meeting the agreed-upon terms and conditions. For example, they might be delivering goods or services that do not meet the specified quality standards or are not delivered within the agreed-upon timeframe; financial discrepancies in invoicing or payments. For example, when a supplier submits an invoice for an amount that does not align with the contracted pricing, the supplier creates a financial issue that needs resolution; contract renewal when a contract may be approaching its expiration date may include discrepancies in the process of renewal. For example, one or more delays in initiating the renewal process or disagreements on the terms for the renewed contract; communication breakdown on issues may arise when there are communication breakdowns between stakeholders. For example, when there is a lack of clarity on certain contract terms, it can lead to misunderstandings and potential disputes; regulatory compliance including changes in regulations or legal requirements may introduce discrepancies in the contract's compliance. Adapting to new regulations or ensuring ongoing compliance can be a challenge.

In some non-limiting embodiments or aspects, a divergence from the established norms, standards, or agreed-upon terms within a contract are deviations that involve: scope changes in the scope of work or deliverables outlined in the contract that were not initially agreed upon. This may include additions, removals, alterations to the originally defined project scope, and/or the like; timeline adjustments including changes to the agreed-upon timelines for project milestones, deliveries, or completion dates. Deviations in the timeline might result from unforeseen circumstances or adjustments to project priorities. Resource allocation including changes in the allocation of resources, such as manpower, technology, or equipment that impact the execution of the contract. This may include a shift in responsibilities or the need for additional resources; quality standards including departures from the quality standards specified in the contract. When the delivered goods or services do not meet the agreed-upon quality levels, it represents a deviation that needs to be addressed; financial adjustments and changes in financial terms, pricing structures, or payment schedules that were not part of the original agreement. This may involve negotiations for additional costs or adjustments to accommodate unforeseen circumstances; regulatory compliance deviations related to changes in regulations or legal requirements that affect the contract. For example, adapting to new compliance standards or ensuring ongoing adherence to regulatory changes may be necessary; contract renewal terms: changes in the terms and conditions for contract renewal that were not anticipated. This may include alterations to pricing, deliverables, or other contractual elements during the renewal negotiation process; and addressing and managing deviations effectively may be essential for successful contract management, and having a system that can identify, evaluate, and guide the resolution of these deviations contributes to the overall efficiency and compliance of the contract lifecycle.

In some non-limiting embodiments or aspects, procurement prediction engine 102 generates a call to action communication. The call to action may be generated or configured for at least one of an intercompany action, external actions, response management, or resolution deployment.

In some non-limiting embodiments or aspects, procurement prediction engine 102 generates or configures a call to action based on at least one of a critical parameter of a cost analysis correlated from operating parameters comprising information about at least one of a usage, a critical parameter of a cost, a critical parameter of an offset, a critical parameter of current product details, or a critical parameter of new product details, a critical parameter of a contract generated with one or more terms, or a critical parameter for one or more identified alternative items.

In some non-limiting embodiments or aspects, a critical parameter for the call to action may be correlated from a plurality of operating parameters defined by one or more of a current contract id, contract description, supplier name, items on contract, start date, end date, product name, item number, catalog number, cost, estimated usage per year, an annualized total cost, product information, product domain, product description, product id, alert detail, alert id, release date/type, alert description, supplier, alert reason, recall level, instructions, additional information, contact information, inventory item id, description, manufacturer item id, vendor item id, commodity contract administrator, clinical resource liaison, requestor, or surgery business office manager. In some non-limiting embodiments or aspects, an agreement type includes at least one of a lease, emergency preparedness, placement, consignment, service, construction, evaluation, GPO, or local pricing.

In some non-limiting embodiments or aspects, procurement prediction engine 102 generates an analysis for at least one of: one or more items of a contract renewal, for one or more items of a contract maintenance, or for one or more purchase requests of a requisition placement in a plurality of accounts of the resource planning system. In some non-limiting embodiments or aspects, an analysis of a contract renewal includes at least one of a cost analysis of the usage, a cost analysis of the cost, a cost analysis of an actual spend, an expected contract value (ECV), a probability of the ECV, a contract of indefinite duration, a one-time contract, a quality analysis, a recalls analysis, a rebid for consumer to consumer, or a department approval, one or more contract terms, supplier information, contact information, training determination, source information including minority owned, existing, or new, stocking information, stocking location, and/or the like.

In some non-limiting embodiments or aspects, an analysis of a contract maintenance for an existing agreement includes at least one of an analysis of one or more calls to action of a cancelation, a price change, or a performance notification.

In some non-limiting embodiments or aspects, procurement prediction engine 102 provides a prediction of a requisition management for one or more products includes at least one of one or more call to actions, including one or more reason codes, one or more services, one or more items, one or more goods, an existing product, product details, or a source strategy assignment.

In some non-limiting embodiments or aspects, the procurement inference engine 102 may be configured to generate an intercompany action. For example, an intercompany action for contract renewal. The intercompany action includes automatically generating a call to action for at least one of: a communication to an end user; a contract type, a department approval, determining a renewal, and if a renew action a request for proposal, an ad hoc approver, an email generation critical parameter including operating parameter template, one or more attachment, and/or the like.

In some non-limiting embodiments or aspects, an intercompany action for a contract maintenance for an existing agreement may automatically generate a draft agreement based on predicted clauses, at least one department approval, at least one preset commodity groups, at least one ad hock approver, or a contract maintenance use template, and/or the like.

In some non-limiting embodiments or aspects, an intercompany action for a requisition management for one or more products includes procurement prediction engine 102 determining when a requisition may be needed, a call to action operating parameter template, one or more attachment, and/or the like.

In some non-limiting embodiments or aspects, procurement prediction engine 102 may be trained to generate an intercompany communication. For example, procurement prediction engine 102 may generate an intercompany communication from operation parameters based on previous communications. For example, previous communications of additional information request, recall notification; recall action needed; recall acknowledgement; product defect acknowledgement received by supply chain, and/or the like.

In some non-limiting embodiments or aspects, procurement prediction engine 102 is configured to generate an external action. In some non-limiting embodiments or aspects, the external action for a renewal contract includes a communication with the supplier. In some non-limiting embodiments or aspects, a communication includes at least one of: a request to the supplier; a request for additional information needed from the supplier; and clearing a call to action to the supplier.

In some non-limiting embodiments or aspects, an external action for a contract maintenance includes a communication with the supplier. For example, the communication may be a request to the supplier; a request for additional information needed from the supplier; clearing a call to action to the supplier, and/or the like.

In some non-limiting embodiments or aspects, the external action for a requisition placement includes a communication with the supplier. As an example, the communication includes a request to the supplier; a request for additional information needed from the supplier and clearing a call to action to the supplier.

In some non-limiting embodiments or aspects, the procurement inference engine is configured to generate a response management action. A response management action for contract renewal includes a communication for at least one communication type. For example, a proposal assessment: emergency care research institute (ECRI) medicine (MD) buyline; new present value; premier; a consumer price index; lease determination; pricing; criminal investigation (CID); an amendment; template documents; redlines; terms of development; innovation; legal; risk management; privacy; vendor signature; requisitions; cost savings and avoidance, a communication for internal approval, including at least one of: an approval to send the external request, a status of a supplier response, including a completed action, a training action, a monitor action, an escalate action, a case closed action; and/or the like.

In some non-limiting embodiments or aspects, response management action for contract maintenance includes a communication for internal approval, including at least one of: an approval to send the external request, a status of a supplier response, including a completed action, a monitor action, an escalate action, a case closed action. In other examples, a response management action for a requisition placement includes a communication for internal approval, including at least one of: an approval to send the external request, a status of a supplier response, including a completed action, a monitor action, an escalate action, a case closed action, and/or the like.

In some non-limiting embodiments or aspects, procurement prediction engine 102 is configured to generate a resolution deployment. In such an example, a resolution deployment for a renewal agreement includes at least one of: an action to activate a contract, an action to monitor a response, an action to escalate a call to action, an action to send a communication or notification, or an action to file information. In another example, a resolution deployment for a contract maintenance for one or more products includes at least one of: an action to activate a contract, an action to monitor a response, an action to escalate a call to action, an action to send a communication or notification, or an action to file information, an action to update a contract, an action to close with no follow up, or an action to close with a threshold number of days to review, and/or the like.

In some non-limiting embodiments or aspects, a requisition placement for one or more products includes at least one of operating parameters: generating a purchase order for the requisition, sending a purchase order, confirming an action is taken, reporting capabilities, or a request for credit, an action to activate a contract, an action to monitor a response, an action to escalate a call to action, an action to send a communication or notification, or an action to file information, closing with no follow up, or closing with a threshold days for follow up.

In some non-limiting embodiments or aspects, procurement prediction engine 102 is configured to automatically deploy a call to action to approvers, the call to action determined. In such an example, procurement prediction engine 102 correlates one or more operating parameters to a critical parameter to determine one or more approvers of a substitute item. Procurement prediction engine 102 may deploy a notification communication to one or more approvers of a substitute item. For example, the notification communication includes at least one of: one or more terms or conditions generated by the procurement inference engine, an original agreement including one or more original terms or conditions provided by a vendor, pricing information, or vendor information.

In some non-limiting embodiments or aspects, the procurement inference engine is configured to generate a close action. A close action for a renewal agreement includes operating parameters. The operating parameters include contract execution: vendor signature; content identifier; approver signature; at least a part of a purchase order; at least a part of a work order; contract not renewal: case is closed; an action to close a purchase order; content identifier closed; contract summary; key terms; or an action to close a case, and/or the like.

In some non-limiting embodiments or aspects, a close action for a contract maintenance includes at least one of a cost savings, a contract summary; one or more key contract terms; or an action to close a case contract summary. In another example, a close action for a requisition placement includes at least one of a cost savings; a contract summary; one or more key contract terms; or an action to close a case contract summary.

In some non-limiting embodiments or aspects procurement prediction engine 102 is configured to automatically monitor approvers for a response to the call to action. In some non-limiting embodiments or aspects, procurement prediction engine 102 receives a response from an approver. In some non-limiting embodiments or aspects, procurement prediction engine 102 determines a response does not approve the item. In some non-limiting embodiments or aspects, procurement prediction engine 102 provides a response, automatically escalates the response to another approver.

In some non-limiting embodiments or aspects, procurement prediction engine 102 automatically escalates. For example, procurement prediction engine 102 correlates one or more operating parameters to a critical parameter to determine at least one escalated approvers of a substitute item. Procurement prediction engine 102 generates a new notification communication with language corresponding to a correlation in the procurement inference engine of the received response with an expected action. Procurement prediction engine 102 deploys a notification communication to one or more escalated approvers of a substitute item which includes information about an expected action.

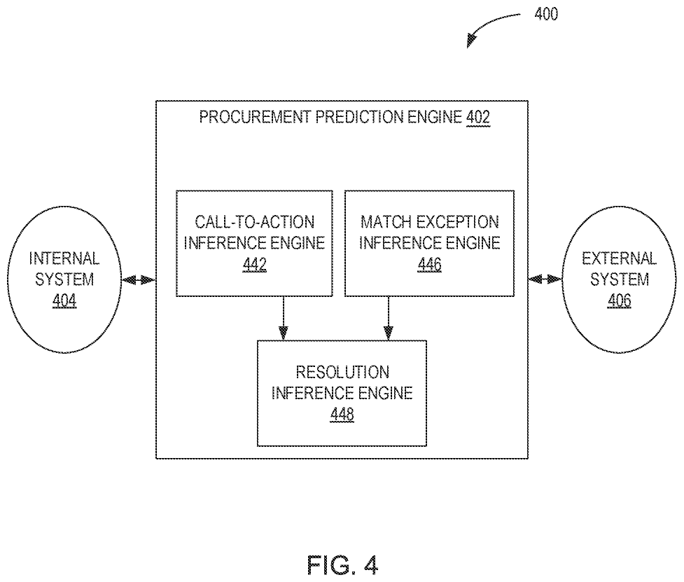

FIG. 4 shows an illustrative computing environment 400 for performing enhanced exception processing using cognitive automation tools in accordance with one or more example embodiments. With reference to FIG. 4, computing environment 400 may include one or more computer systems. For example, computing environment 400 may include a procurement prediction engine 402, internal system 404, external system 406, call-to-action inference engine 442, match exception inference engine 446, and resolution inference engine 448. In some non-limiting embodiments, one or more of the components of computing environment 400 are performed (e.g., completely, partially, etc.) by an procurement prediction engine 402 (e.g., one or more devices of procurement prediction engine 402, one or more processors of the procurement prediction engine 402, one or more CPU or GPU of procurement prediction engine 402, one or more processors of procurement prediction engine 102, etc.), resource planning system 104 (e.g., one or more devices of exception resource planning system 104), data visualization system 106 (e.g., one or more devices of data visualization system 106), user computing device 112 (e.g., one or more devices of user computing device 112), external computing system 118 (e.g., one or more devices of external computing system 118), and supplier sales automation 120 (e.g., one or more devices of supplier sales automation 120). For example, in some non-limiting embodiments or aspects, internal system 404 includes resource planning system 104 such as ERP systems from PeopleSoft, Oracle, Epicor, SAP, and/or the like. In some examples, external system 406 includes supplier sales automation system 120, Salesforce.com, NetSuite, PipeDrive, and/or the like. Internal system 404 and external system 406 may be interconnected by the procurement prediction engine 102 to optimize the supply chain.

In some non-limiting embodiments or aspects, call-to-action inference engine 442 transforms predictions generated by procurement prediction engine 402 to determine appropriate call-to-actions. For example, when procurement prediction engine 402 identifies a specific exception like “voucher's extended price exceeds the purchase order's extended price,” call-to-action inference engine 442 may activate actions like reviewing the price discrepancy, investigating further, possibly alerting relevant personnel, and/or the like. In some non-limiting embodiments or aspects, call-to-action inference engine 442 is trained to generate a call-to-action.

In some non-limiting embodiments or aspects, a mismatch between voucher and PO prices is detected within tolerance limits. In such an example, call-to-action inference engine 442 generates a call in cases where the price percentage tolerance for example may not equate to zero, and the converted voucher price may be beyond the calculated PO price range. In such an example, call-to-action inference engine 442 activates a call for a thorough review of pricing agreements and terms. Likewise, call-to-action inference engine 442 determines call-to-actions for scenarios where unit price tolerance deviations and anomalies involving RTV or credit adjustment amount exceed the PO matched amount. Call-to-action inference engine 442 activate a call including one or more relevant (e.g., related, etc.) recommendations for resolution.

In some non-limiting embodiments or aspects, when the total vouchered quantity surpasses the allowed quantity considering both PO quantity and over-receiving allowance, call-to-action inference engine 442 may activate a call for adjusting the quantities, investigating over-receiving reasons, or verifying purchase orders. In some examples, call-to-action inference engine 442 activates a call for situations where receiving percentage tolerance deviations lead to excess vouchered quantity.

In some non-limiting embodiments or aspects, call-to-action inference engine 442 predicts discrepancies (e.g., exceptions, etc.) In total vouchered amount exceeding the PO amount, call-to-action inference engine 442 might activate a call for verifying invoicing and pricing.

In some non-limiting embodiments or aspects, call-to-action inference engine 442 predicts pricing discrepancies. For example, when pricing mismatches are identified between the initial concept and the formal contract, call-to-action inference engine 442 may initiate a call for a comprehensive review of these pricing disparities. In such an example, call-to-action may include conducting a thorough examination of the pricing components to determine the root causes of the discrepancies. The goal is to align the contract terms with the original concept and ensure pricing accuracy.

In some non-limiting embodiments or aspects, call-to-action inference engine 442 negotiates contract terms. For example, when discrepancies emerge in the contract terms compared to the initial concept, the call-to-action inference engine plays a pivotal role in initiating contract renegotiations. It may activate calls for negotiations with relevant parties, such as suppliers or stakeholders, to reconcile the terms and reach a consensus that aligns the contract with the initial concept.

In some non-limiting embodiments or aspects, call-to-action inference engine 442 generates an alert to relevant stakeholders. For example, when variations between the concept and contract are detected, call-to-action inference engine 442 is capable of activating calls to alert pertinent stakeholders. In some examples, the alerts serve to notify individuals or teams involved about the identified discrepancies, ensuring that everyone is informed and can take appropriate actions.

In some non-limiting embodiments or aspects, call-to-action inference engine 442 verifies purchase orders. For example, when deviations are determined (e.g., exist, etc.) between the purchase orders and the original concept, call-to-action inference engine 442 may initiate calls to verify and align these purchase orders with the agreed-upon contract terms. This verification process ensures that the purchase orders accurately reflect the contracted requirements.

In some non-limiting embodiments or aspects, call-to-action inference engine 442 investigates price deviations. For example, for scenarios where price discrepancies are observed, call-to-action inference engine 442 activates calls to investigate the underlying causes of these deviations. Call-to-action inference engine 442 can generate a comprehensive prediction of market prices, supplier agreements, and other factors contributing to the pricing differences, ensuring transparency in pricing matters.

In some non-limiting embodiments or aspects, call-to-action inference engine 442 may adjust quantities. For example, call-to-action inference engine 442 may detect discrepancies in quantities between the contract and the initial concept. Call-to-action inference engine 442 may activate calls for quantity adjustments. These actions focus on bringing the contracted quantities in line with the actual requirements, promoting efficient resource management.

In some non-limiting embodiments or aspects, call-to-action inference engine 442 reevaluates supplier agreements. For example, when discrepancies are identified in supplier agreements compared to the concept, call-to-action inference engine 442 may activate calls to reevaluate these agreements. In such an example, call-to-action inference engine 442 may call for a comprehensive assessment of the agreements to ensure that they align with the contract terms and the concept's expectations.

In some non-limiting embodiments or aspects, call-to-action inference engine 442 may reconcile payment terms. For example, the call-to-action inference engine activates calls to reconcile payment term differences when variations between the concept and contract arise. In some examples, during the reconciliation process call-to-action inference engine 442 determines that the payment terms in the contract are consistent with the original concept, fostering financial clarity.

In some non-limiting embodiments or aspects, call-to-action inference engine 442 monitors performance. For example, call-to-action inference engine 442 maintains continuous monitoring of supplier performance to uphold contractual agreements. Call-to-action inference engine 442 may initiate calls to monitor supplier performance, ensuring that it aligns with the terms outlined in the contract. In this way, monitoring helps maintain consistent and reliable supplier performance.

In some non-limiting embodiments or aspects, call-to-action inference engine 442 validates compliance. For example, call-to-action inference engine 442 verifies compliance with regulatory requirements and internal policies that are critical in the concept-to-contract process. Call-to-action inference engine 442 may activate calls to validate compliance, ensuring that both the concept and the contract adhere to necessary standards, rules, and regulations. In this way, call-to-action inference engine 442 promotes legal and ethical adherence in the supply chain.

In some non-limiting embodiments or aspects, match exception inference engine 446 may obtain observed match exceptions to provide more context and insights into other match exceptions. For example, based on procurement prediction engine 402 predicting that a voucher's extended price exceeds a threshold tolerance, match exception inference engine 446 may obtain a detailed breakdown (e.g., a specification, etc.) of how the calculations were made, showcasing the extent of the discrepancy and the parameters that triggered the prediction.

In some non-limiting embodiments or aspects, resolution inference engine 448 may provide automated suggestions for resolving the predicted exceptions. Resolution inference engine 448 may obtain historical or observed data and historical or observed resolutions for learning potential solutions for resolving match exceptions. For example, when a price discrepancy is predicted, resolution inference engine 448 may recommend initiating a price negotiation with the supplier, verifying the data input, or recalculating the tolerances.

The engines (e.g., collectively, procurement prediction engine 402, call-to-action inference engine 442, match exception inference engine 446, and resolution inference engine 448) form an integrated system that enhances item processing, automates decision-making, and optimizes distribution within the healthcare supply chain.

In some non-limiting embodiments or aspects, the engines utilize machine learning techniques to analyze historical data, identify patterns, and generate prediction models tailored to different accounts, suppliers, or transactions. The engines apply supervised and unsupervised learning to predict exceptions and inaccurate activities within the healthcare supply chain. The engines generate model representations of the predicted exceptions, accounts, payment information, the parties included, and other relevant data to form insights about new observations. In this way, engines provide or generate predictive actions and interpretations for enhancing the accuracy of decision-making related to the exceptions. The engines facilitate integration, for example by establishing efficient integration and communication between supply chain systems, improving interactions with enterprise resource systems, database management systems, workflow systems, and sales automation systems. The engines coordinate the transmission of supply information within a call-to-action system for timely and effective communication regarding steps for resolving the exception across the supply chain. The engines may interact with one or more of resource planning systems, data visualization systems, other enterprise systems, and/or the like, to provide seamless (e.g., coordinated, etc.) data exchange and collaboration. The engines may create a dynamic system that not only predicts exceptions but also guides stakeholders, for example, with specified actions for advancing towards appropriately resolving these exceptions.

In some non-limiting embodiments or aspects, a company's procurement prediction engine 402 continually monitors incoming invoices and compares them to the corresponding goods received. It identifies a match exception where the invoice quantity doesn't match the received quantity.

In some non-limiting embodiments or aspects, the inference engine within procurement prediction engine 402 categorizes this match exception as “receiving—quantity mismatch” based on the established patterns and relationships between attributes.

In some non-limiting embodiments or aspects, call-to-action inference engine 442 plays a pivotal role in translating the predicted match exceptions into actionable inferences within the healthcare supply chain. In some examples, the described scenarios include various types of exceptions, such as price, ltd, receiving, and PO status anomalies.

In some non-limiting embodiments or aspects, procurement prediction engine 402 identifies that a voucher's extended price exceeds the purchase order's extended price. In such an example, the extended price tolerance is not equal to zero. In such an example, call-to-action inference engine 442 may activate call-to-actions such as reviewing the price discrepancy, investigating the reason behind the excess amount, alerting relevant personnel or stakeholders.

In some non-limiting embodiments or aspects, a mismatch between voucher and PO prices is detected within tolerance limits. In such an example, call-to-action inference engine 442 generates a call in cases where the price percentage tolerance for example may not equate to zero, and the converted voucher price may be beyond the calculated PO price range. In such an example, call-to-action inference engine 442 activates a call for a thorough review of pricing agreements and terms. In some examples, call-to-action inference engine 442 determines call-to-actions for scenarios where unit price tolerance deviations and anomalies involving RTV or credit adjustment amount exceed the PO matched amount. Call-to-action inference engine 442 activate a call including one or more relevant (e.g., related, etc.) recommendations for resolution.

In some non-limiting embodiments or aspects, when the total vouchered quantity surpasses the allowed quantity considering both PO quantity and over-receiving allowance, call-to-action inference engine 442 may activate a call for adjusting the quantities, investigating over-receiving reasons, or verifying purchase orders. In some examples, call-to-action inference engine 442 activates a call for situations where receiving percentage tolerance deviations lead to excess vouchered quantity.

In some non-limiting embodiments or aspects, call-to-action inference engine 442 predicts discrepancies (e.g., exceptions, etc.) in total vouchered amount exceeding the PO amount, call-to-action inference engine 442 might activate a call for verifying invoicing and pricing.

In some non-limiting embodiments or aspects, call-to-action inference engine 442 generates recommendations for extended price percentage and tolerance deviations, along with activating a call for handling mismatched quantities, that may be provided by the engine.

In some non-limiting embodiments or aspects, call-to-action inference engine 442 activates a call based on discrepancies between packing slips on voucher and receiver lines. For example, call-to-action inference engine 442 activates a call for comparing the received goods to the associated documentation. When the matching process can't locate receipts for a specific purchase order on the voucher line, call-to-action inference engine 442 activates a call for investigating the status of the missing receipts and assessing the impact on processing.

For voucher line amounts exceeding the combined receiver line amounts, call-to-action inference engine 442 activates a call for reviewing the discrepancy and ensuring accuracy in calculations.

Similarly, for issues with total received quantity exceeding the accepted receiver quantity, call-to-action inference engine 442 activates a call for reconciling the discrepancies and making necessary adjustments.

In some non-limiting embodiments or aspects, when payment terms, supplier information, or item detail mismatches occur between various documents, call-to-action inference engine 442 activates a call for verifying the accuracy of data and taking corrective actions.

In each example, call-to-action inference engine 442 obtains the predictions, evaluates the implications, and recommends appropriate actions. These recommendations may include investigating further, initiating communications with relevant parties, adjusting quantities, verifying data accuracy, and addressing discrepancies. The engine serves as a valuable tool for efficient decision-making, process optimization, and seamless collaboration across the healthcare supply chain.

Resolution inference engine 448 is configured to obtain historical data (e.g., review features of the historical data as described herein, etc.) and resolutions (e.g., review features of resolved match exceptions as described herein, etc.) to provide automated context for addressing one or more other identified exceptions. For example, in the context of various exception types, including Price, LTD, Receiving, and PO Status anomalies (e.g., exceptions, etc.).

In some non-limiting embodiments or aspects, resolution inference engine 448 may detect a situation where a voucher's extended price exceeds the purchase order's extended price. Resolution inference engine 448 may recommend actions for resolving the discrepancy (e.g., exceptions, etc.).

Resolution inference engine 448 initiates a price negotiation with the supplier. Resolution inference engine 448 verifies the accuracy of data input for both the voucher and the purchase order. Resolution inference engine 448 recalculates the tolerances and compares them with the actual values.

Significant disparities exist between the initial concept and the formal contract. In such an example, resolution inference engine 448 may generate a viable resolution by amending the contract to align it with the original concept. This involves revising and updating the contract terms, conditions, and specifications to accurately reflect the concept.

In some non-limiting embodiments or aspects, resolution inference engine 448 renegotiates terms. In such an example, resolution inference engine 448, when contract terms require adjustments to match the concept, stakeholders may choose to renegotiate the terms with suppliers or other parties involved. This resolution aims to ensure that all parties are in agreement with the terms and conditions outlined in the contract.

In some non-limiting embodiments or aspects, resolution inference engine 448 clarifies ambiguities. In such an example, resolution inference engine 448 determines cases where ambiguities or misunderstandings arise. Resolution inference engine 448 generates, approves, or requests change language clarifying any vague or unclear sections of the contract, and or the like. In this way, resolution inference engine 448 ensures that all parties have a shared understanding of the terms and expectations.

In some non-limiting embodiments or aspects, resolution inference engine 448 mediates or arbitrates. When disputes between parties hinder the alignment of the contract with the concept, mediation or arbitration can be employed as a resolution. An impartial third party is called in to facilitate discussions and help parties reach a fair and mutually acceptable agreement.

In some non-limiting embodiments or aspects, resolution inference engine 448 reverts to the original concept. In certain situations, it may be appropriate to revert back to the original concept when it is deemed more practical or suitable than the existing contract. In such an example, resolution inference engine 448 abandons the current contract and reinstates the initial concept.

In some non-limiting embodiments or aspects, resolution inference engine 448 implements change orders. For example, resolution inference engine 448 determines changes are necessary to align the contract with the concept, a resolution can involve the creation and implementation of change orders. In such an example, resolution inference engine 448 implements change orders which document the modifications to the contract and ensure that the changes are formally acknowledged and accepted by all parties.

In some non-limiting embodiments or aspects, resolution inference engine 448 reworks resource allocation. For example, resolution inference engine 448 determines discrepancies involve resource allocation. In such an example, resolution inference engine 448 may include the reevaluation and adjustment of resource allocation plans to better align with the concept's requirements.

In some non-limiting embodiments or aspects, resolution inference engine 448 aligns supplier agreements. For example, resolution inference engine 448 determines supplier agreements differ from the concept, the resolution may involve aligning these agreements with the concept's expectations. In such an example, resolution inference engine 448 may update supplier contracts or agreements to ensure they are consistent with the overarching concept.

In some non-limiting embodiments or aspects, resolution inference engine 448 reevaluates payment terms. For example, resolution inference engine 448 determines payment terms deviate from the concept. In such an example, resolution inference engine 448 provide reevaluation and adjustment of payment terms to better align with the concept and the contractual requirements.

In some non-limiting embodiments or aspects, resolution inference engine 448 enhances quality control. For example, resolution inference engine 448 determines quality discrepancies between the concept and the contract that require a resolution. In such an example, resolution inference engine 448 enhances quality control processes to ensure that the final product or service aligns with the concept's quality standards.

In some non-limiting embodiments or aspects, the total vouchered quantity exceeds the allowed quantity or a vouchered amount surpasses the PO amount. In such an example, resolution inference engine 448 determines that the total vouchered quantity exceeds the allowed quantity or a vouchered amount surpasses the PO amount. The resolution inference engine 448 may offer steps to perform. For example, resolution inference engine 448 activates reviewing the over-receiving allowance and adjusting quantities as necessary.

In some non-limiting embodiments or aspects, resolution inference engine 448 activates a reconciliation of quantities and amounts. Resolution inference engine 448 receives exceptions.

For discrepancies such as packing slip mismatches or issues with identifying receipts, resolution inference engine 448 activates an action for verifying packing slip data and comparing it with the receiver line. In some non-limiting embodiments or aspects, resolution inference engine 448 activates investigating receipt status and locating missing or unmatched receipts.

For PO status exceptions, such as in cases of differences in payment terms codes or supplier information, resolution inference engine 448 may provide recommendations to recipients recommending steps for resolving the exception. For example, resolution inference engine 448 may activate a call for ensuring consistent and accurate information across all related documents. Also, resolution inference engine 448 may activate a call for initiating communication with the relevant parties to resolve discrepancies.

Resolution inference engine 448 may leverage its analysis of historical resolutions to provide context-specific recommendations. For example, when a specific price discrepancy is similar to past cases that were resolved through a certain action, the engine may suggest that action as a potential solution. This process helps streamline decision-making and reduces the time needed to address exceptions.

Match exception inference engine 446 is an integral component designed to enhance the process of determining match exceptions by leveraging predicted exceptions and providing more contextual insights. Operating within the healthcare supply chain, this engine plays a key role in analyzing and understanding the predictions made by the procurement prediction engine 402, thus enabling a more informed approach to identifying and handling exceptions. In the context of specific scenarios involving Price, LTD, Receiving, and PO Status anomalies, match exception inference engine 446 operates on factors of the exception (e.g., features of the information, etc.).

When procurement prediction engine 402 predicts a particular exception, such as a voucher's extended price exceeding the purchase order's extended price, match exception inference engine 446 responds to a call to provide comprehensive context. For example, match exception inference engine 446 can retrieve a detailed breakdown of the calculations that led to the prediction. In some examples, a breakdown showcases the specific parameters and data points that contributed to the predicted discrepancy. In other examples, it may provide the specific parameters and data points that contributed to a problem associated with the predicted discrepancy. In still other examples, match exception inference engine 446 obtains the underlying factors that triggered the prediction and provides explanation of the extent of the deviation.

In some non-limiting embodiments or aspects, procurement prediction engine 402 utilizes these insights to match the features of the current prediction to historical cases. In some examples, procurement prediction engine 402, by identifying patterns and similarities between past and present situations, may generate (e.g., make, etc.) new predictions based on accumulated knowledge. This capability improves the accuracy of predictions.

In some non-limiting embodiments or aspects, procurement prediction engine 402 may determine one or more price exceptions. As an example, when procurement prediction engine 402 forecasts a situation where a voucher's extended price exceeds the purchase order's extended price, match exception inference engine 446 may display a detailed analysis of how one or more calculations were performed. In some examples, match exception inference engine 446 may also highlight the relevant tolerance thresholds and deviations, such that the information guides users in understanding the factors behind the predicted exception.

Match exception inference engine 446 provides capabilities to leverage historical data and insights for improving the accuracy and the relevance of predictions. By continuously learning from past cases (e.g., data or other information associated with past cases, etc.) and adapting to new scenarios, the engine contributes to a more proactive and informed approach to managing match exceptions within the healthcare supply chain.

In some non-limiting embodiments or aspects, match exception inference engine 446 reviews data when predicting exceptions, and places exceptions as they are classified into sub-classes based on root causes. In some examples, by referencing established standard operating procedures (SOPs) created by the contracts and procurement team, resolution inference engine 448 may provide users with actionable insights which align with best practices. For example, call-to-action comms (e.g., communications, messages, requests, instructions, etc.) guide users on how to address unique circumstances represented by each sub-class, thereby ensuring efficient handling of purchase orders and invoices. Moreover, resolution inference engine 448 satisfies a crucial role in improving the healthcare supply chain to promptly and effectively respond to match exceptions while minimizing disruptions.

In some non-limiting embodiments or aspects, procurement prediction engine 402 identifies the appropriate recipients for a specific match exception. In this example, the recipients may include the internal warehouse department responsible for managing received goods and the AP team that handles invoice discrepancies.

In some non-limiting embodiments or aspects, after the match exception is classified and recipients determined, procurement prediction engine 402 activates a call-to-action. This call-to-action is configured to execute each step (e.g., one or more specific steps to be taken to address the “Receiving—Quantity Mismatch” match exception, etc.).

In some non-limiting embodiments or aspects, an automated communication is triggered and sent to one or more designated recipients. For example, procurement prediction engine 402 generates an email automatically and sends it to a warehouse department and the AP team. The email may include information about the match exception and details about the quantity discrepancy (in the example), and includes a link to access relevant documents like the invoice and the receiving report.

In some non-limiting embodiments or aspects, procurement prediction engine 402 communicates to the recipient the automated communication which provides instructions to the recipient with steps for the prescribed action. The steps are programmed or configured to lead to resolution. In such an example, the warehouse team may receive a communication identifying the received goods and/or a request to verify the actual quantity, while the AP team examines the invoice. When the discrepancy is confirmed, the teams are further coordinated by the procurement prediction engine for further actions to resolve the issue. For example, instructions are sent for either adjusting the payment or by coordinating with the supplier to correct the invoice.

In some non-limiting embodiments or aspects, communication channels for various types of recipients include organization's internal communication platform, such as a collaboration tool or intranet, for the internal AP department to receive notifications within their work environment. External suppliers may be communicated with through email, while the SCM team might use a dedicated SCM platform or tool. Warehouse personnel may receive notifications via email and an internal notification system. For requesters or internal departments included in procurement, collaboration tools or internal messaging systems might be used. Management or directors may receive notifications through email or a dedicated executive communication channel. External partners or vendors might access communications through a secure supplier portal. The data visualization team may be informed via internal communication tools or project management platforms. Examples of chosen communication channels include email, internal communication platforms like Slack or Microsoft Teams, messaging apps such as WhatsApp, intranet postings, dedicated SCM systems, supplier portals, SMS notifications, automated phone calls, and notifications within business applications. The choice of communication channel depends on recipient preferences, message urgency, match exception nature, and the organization's existing communication infrastructure.

In some non-limiting embodiments or aspects, as the resolution progresses, the status of the match exception may change automatically within the company's system. Depending on the progress, procurement prediction engine 402 may activate follow-up communications to keep relevant parties informed.

In some non-limiting embodiments or aspects, when procurement prediction engine 402 determines that the match exception remains unresolved for a certain duration, procurement prediction engine 402 triggers the escalation process described above. For example, when the issue persists for more than a week, procurement prediction engine 402 automatically escalates the case to higher management levels for attention. As an example, procurement prediction engine 402 automatically escalates the case to higher management levels by activating a call-to-action for automated communication which provides instructions to the recipient's higher management levels with steps for the prescribed action, configured to lead to resolution. In this example, the match exception “Receiving—Quantity Mismatch” triggers an automated communication to the appropriate recipients, guiding them on how to address the issues for efficiently resolving the exception. This process minimizes processing and accessing data resources, accelerates exception resolution, and enables accurate collaboration with timely messages among relevant departments.

In some non-limiting embodiments or aspects, “Quick Invoices” as used herein refer to a category of invoices that the system does not know how to handle automatically. These quick invoices may not be processed through the regular matching workflow because they lack certain information or have characteristics that prevent the system from automatically determining the appropriate actions for them. Instead, they require manual review and intervention by users to be processed correctly.

In some non-limiting embodiments or aspects, quick invoices as referred to herein may not include factors suitable to address situations where the automated matching process cannot determine how to match an invoice with corresponding purchase orders or receiving information accurately. In some examples, match exception inference engine 446 determines an invoice might be categorized as a quick invoice based on the factors. For example, match exception inference engine 446 determines incomplete data, for example, the invoice may have missing or insufficient data fields required for the matching process, such as missing purchase order numbers, vendor details, or other critical information.

In some non-limiting embodiments or aspects, match exception inference engine 446 may determine unrecognized vendors: For example, match exception inference engine 446 determines that the system may not include the vendor information (e.g., in its database, data store 108, etc.) leading to uncertainty about how to process the invoice. In another example, match exception inference engine 446 may predict that the items listed on the invoice may not have corresponding records in the system, making it challenging for the system to automatically match them to existing purchase orders or receipts. In another example, based on non-standard formats, match exception inference engine 446 may predict that some invoices may not follow the standard formats expected by the system, making it difficult for automated algorithms to interpret and match the data accurately.

In some non-limiting embodiments or aspects, match exception inference engine 446 generates, determines, and activates a call for quick invoices that require human intervention and cannot be processed automatically through the regular invoice matching workflow due to incomplete or challenging data. The call-to-action is used to ensure accurate and proper handling of invoices that cannot be immediately matched by the system.

Match exception inference engine 446 may identify quick invoices based on specific criteria, such as missing or incomplete data fields, unrecognized vendors, or any other characteristic that indicates the system does not know how to handle the invoice. Quick invoices may be flagged and separated from the regular invoice processing flow to prevent them from proceeding to matching until further action is taken. The system may generate alerts or notifications to relevant users or teams to review and manually handle these quick invoices.

In some non-limiting embodiments or aspects, the system may analyze the match exception and relevant data to identify potential alternate POs that may resolve the exception. By searching historical data and related transactions, the system can suggest other POs that may be associated with the invoice, thereby offering a possible corrective action.

In some non-limiting embodiments or aspects, the procurement prediction engine may provide a user interface or automated functionality for transmitting images to users to review and select the appropriate alternate PO for resolution.

In some non-limiting embodiments or aspects, various technologies such as rules engines, classification engines, and data analysis to implement these steps include a combination of automated processes, user interfaces for manual review and resolution, and communication functionalities to inform users about invoices and facilitate collaboration in handling match exceptions.

In some non-limiting embodiments or aspects, the procurement prediction engine 402 is engaged to extract data and identify exceptions stemming from issues within requisition processes. Match exception inference engine 446 may identify exceptions within workflow tasks and approvals. Match exception inference engine 446 facilitates seamless data exchange through data integration, connecting with the internal system and triggering external activities. For example, a call to action for a user to access the supplier database enables the system to retrieve supplier information, ensuring efficient vendor relationship management.

Procurement prediction engine 402 engine optimizes the sourcing process, encompassing supplier selection and proposal management. Salesforce's workflow automation capabilities enable the progression of tasks based on predefined rules and conditions.

Procurement prediction engine 402 communication interface ensures the smooth exchange of relevant data and updates between internal and external systems. Inferences and reporting functions within the supply chain to facilitate tracking of sourcing activities and performance metrics.

APIs or Middleware establish seamless integration and synchronization of data between internal and external systems. Data exchange functionalities ensure the smooth transfer of requisition details, supplier information, and contract-related data. Notifications and alerts guarantee timely communication with stakeholders to prompt necessary actions.

In some non-limiting embodiments or aspects, procurement prediction engine 402 orchestrates the creation of new items for reorder, substitute, restock, or recall by orchestrating parallel pipelines in both the internal and external systems. It adeptly manages enterprise activities through workflow automation and adeptly interprets operating parameters in correlation with critical factors. Furthermore, procurement prediction engine 402 proficiently diagnoses and juxtaposes operating parameters with predefined threshold values, efficiently tailoring responses, communications, or actions based on these correlations. In some examples, predictive capabilities enable proactive actions, while precision in inventory management and the elimination of inaccuracies enhance system effectiveness. The engine seamlessly automates actions within healthcare supplier systems and optimizes value analysis and item management. Within the ERP HR component, it manages employee records, benefits, and payroll. This holistic approach operates within an interconnected computing environment, elevating overall system efficiency and efficacy.

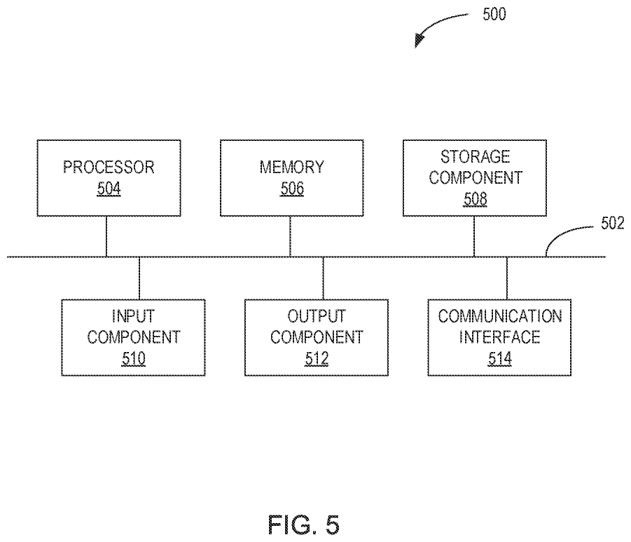

Referring now to FIG. 5, FIG. 5 is a diagram of example components of device 500. Device 500 may correspond to computing environment 100, and may include a procurement prediction engine 102 (e.g., one or more devices of procurement prediction engine 102), resource planning system 104 (e.g., one or more devices of exception resource planning system 104), data visualization system 106 (e.g., one or more devices of data visualization system 106), data store 108, user computing device 112 (e.g., one or more devices of user computing device 112), external computing system 118 (e.g., one or more devices of external computing system 118), and supplier sales automation 120 (e.g., one or more devices of supplier sales automation 120).

As shown in FIG. 5, device 500 may include bus 502, processor 504, memory 506, storage component 508, input component 510, output component 512, and communication interface 514. Bus 502 may include a component that permits communication among the components of device 500. In some non-limiting embodiments or aspects, processor 504 may be implemented in hardware, software, or a combination of hardware and software. For example, processor 504 may include a processor (e.g., a CPU, a GPU, an accelerated processing unit (APU), and/or like), a microprocessor, a digital signal processor (DSP), and/or any processing component (e.g., a field-programmable gate array (FPGA), an application-specific integrated circuit (ASIC), and/or the like), and/or the like, which can be programmed to perform a function. Memory 506 may include random access memory (RAM), read-only memory (ROM), and/or another type of dynamic or static storage device (e.g., flash memory, magnetic memory, optical memory, and/or like) that stores information and/or instructions for use by processor 504.

Storage component 508 may store information and/or software related to the operation and use of device 500. For example, storage component 508 may include a hard disk (e.g., a magnetic disk, an optical disk, a magneto-optic disk, a solid state disk, and/or like), a compact disc (CD), a digital versatile disc (DVD), a floppy disk, a cartridge, a magnetic tape, and/or another type of computer-readable medium, along with a corresponding drive.

Input component 510 may include a component that permits device 500 to receive information, such as via user input (e.g., a touch screen display, a keyboard, a keypad, a mouse, a button, a switch, a microphone, a camera, and/or like). Additionally or alternatively, input component 510 may include a sensor for sensing information (e.g., a global positioning system (GPS) component, an accelerometer, a gyroscope, an actuator, and/or like). Output component 512 may include a component that provides output information from device 500 (e.g., a display, a speaker, one or more light-emitting diodes (LEDs), and/or the like).

Communication interface 514 may include a transceiver (e.g., a transceiver, a receiver and transmitter that are separate, and/or the like) that enables device 500 to communicate with other devices, such as via a wired connection, a wireless connection, or a combination of wired and wireless connections. Communication interface 514 may permit device 500 to receive information from another device and/or provide information to another device. For example, communication interface 514 may include an Ethernet interface, an optical interface, a coaxial interface, an infrared interface, a radio frequency (RF) interface, a universal serial bus (USB) interface, a Wi-Fi® interface, a Bluetooth® interface, a Zigbee® interface, a cellular network interface, and/or the like.

Device 500 may perform one or more processes described herein. Device 500 may perform these processes based on processor 504 executing software instructions stored by a computer-readable medium, such as memory 506 and/or storage component 508. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 506 and/or storage component 508 from another computer-readable medium or from another device via communication interface 514. When executed, software instructions stored in memory 506 and/or storage component 508 may cause processor 504 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

The number and arrangement of components shown in FIG. 5 are provided as an example. In some non-limiting embodiments or aspects, device 500 may include additional components, fewer components, different components, or differently arranged components than those shown in FIG. 5. Additionally or alternatively, a set of components (e.g., one or more components) of device 500 may perform one or more functions described as being performed by another set of components of device 500.

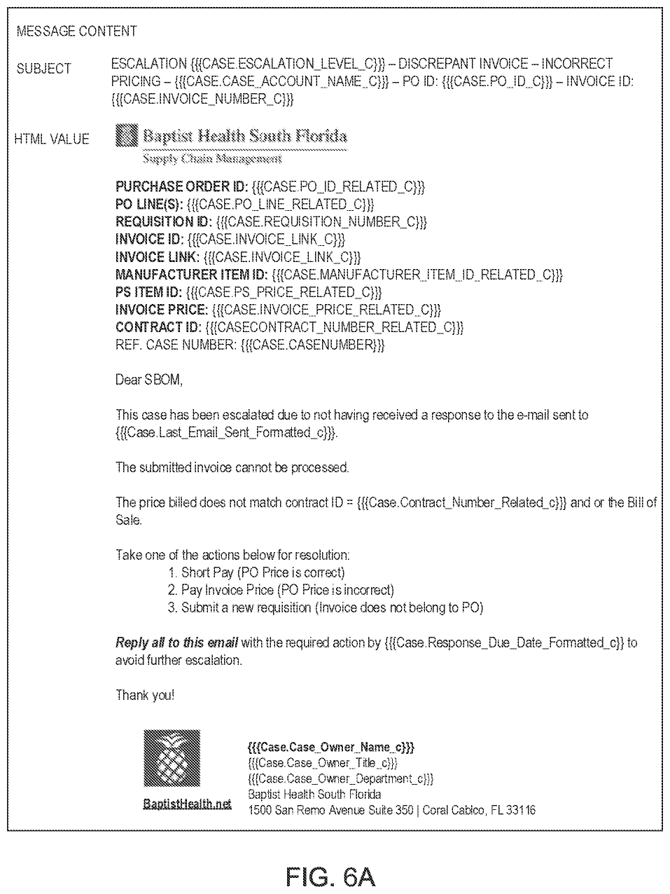

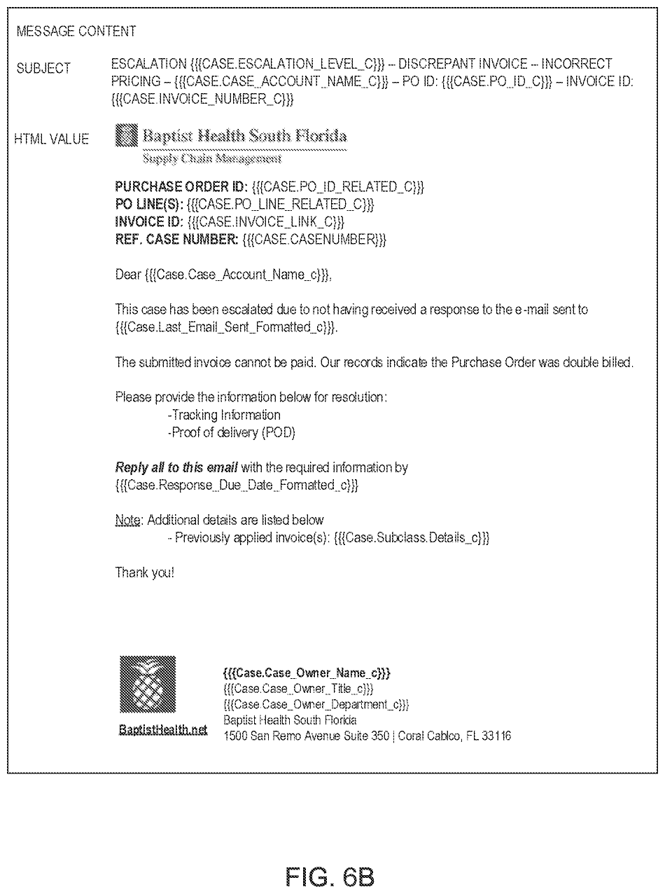

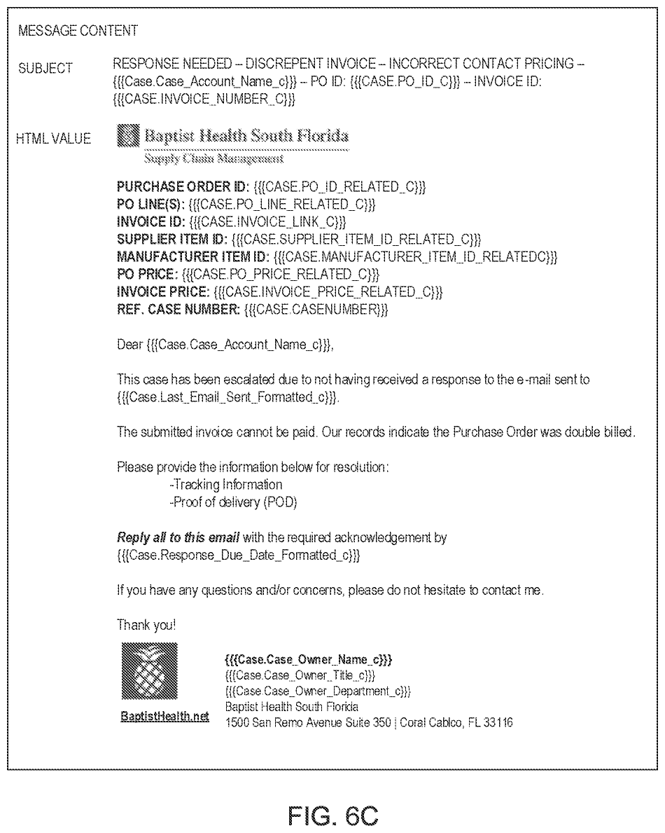

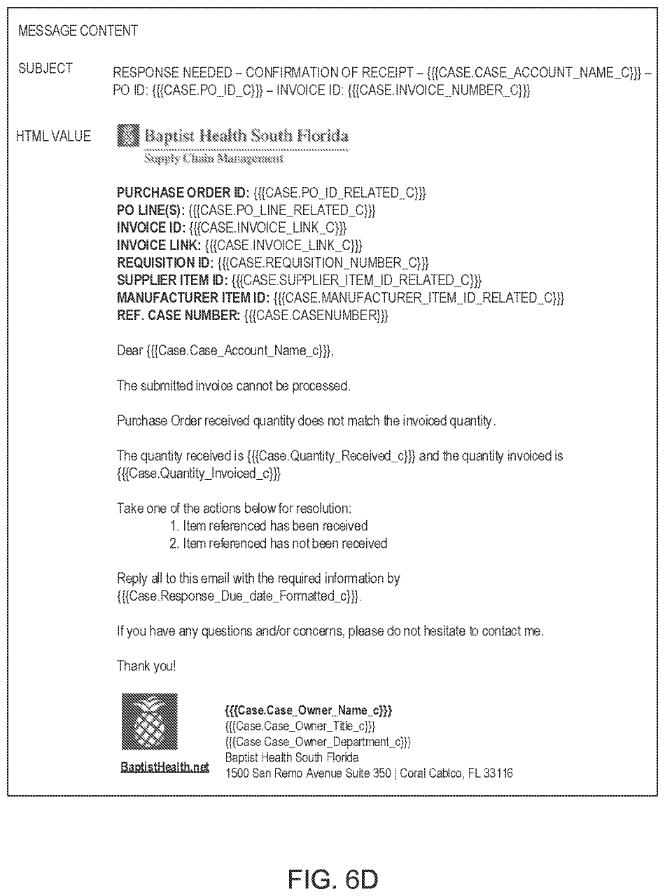

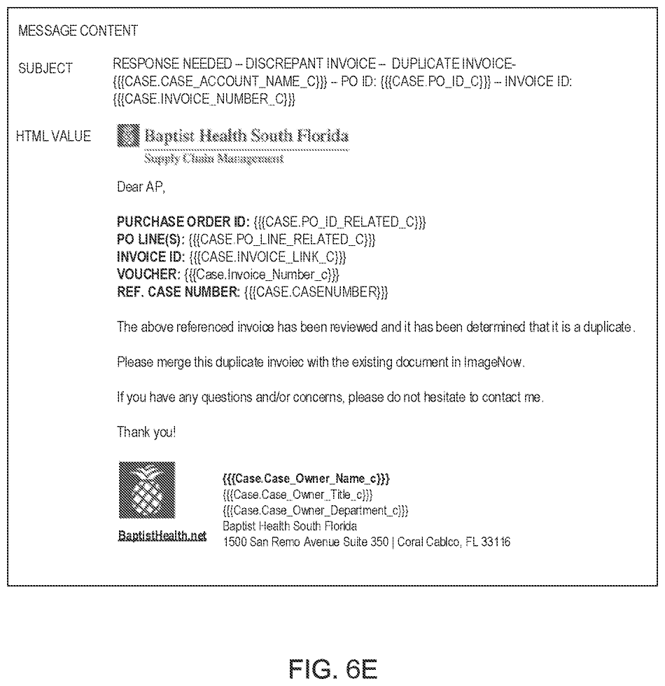

With reference to FIGS. 6A-6E, FIGS. 6A-6E are exemplary call-to-action illustrations for communicating in a supply chain according to non-limiting embodiments. For example, C2C prediction engine 102 notification with a call-to-action statement to recipient for prompting a user to act on an aspect of a match exception.

In some non-limiting embodiments or aspects, managing invoice discrepancies includes an automated workflow that ensures accurate and timely case creation, resolution, and communication. This system operates through daily integration data files, where new rows of data trigger the creation of corresponding cases. All unresolved invoice discrepancies are displayed within the interface until they are resolved, and their presence in the interface corresponds to their availability in the integration data files. Rules automatically assign cases during their creation, and cases are closed when the associated invoice discrepancies have been resolved and are no longer present in the integration data files.

In some non-limiting embodiments or aspects, the engines determine the appropriate email template for outbound communication. This process initiates an automatic outbound case email that is directed to the relevant recipient(s). If a case lacks sufficient contact information, the automatic email transmission will fail. In such cases, the user is notified, the automatic email sent field remains unchecked, and the Error Message field is populated with relevant error details.

In some non-limiting embodiments or aspects, responding to inbound emails activate changes in case status, with a logged inbound email response causing the case status to transition to “pending agent review.” An email notification of this change is sent to the case owner. As match exceptions are resolved, salesforce automatically defaults the resolution status if not already provided by the case owner. However, end users are required to manually update this status, accompanied by a resolution comment. In accordance with predefined timeframes, cases are automatically escalated, and corresponding emails are sent to the case owner's leader. Escalation takes place when a specified number of days pass since the last email requiring a response was recorded in the case. This process results in the system updating relevant case fields pertaining to the escalation.

In some non-limiting embodiments or aspects, intelligent responses enhance communication by incorporating action buttons into case-related emails. These action buttons allow recipients to respond without directly replying to the email. For instance, a price discrepancy email could contain action buttons like “will correct invoice to use PO price” and “researching price discrepancy.” To safeguard accuracy, out-of-office responses are identified and handled separately, thus excluding them from valid responses.

In some non-limiting embodiments or aspects, escalations are made when outgoing emails marked with a response requirement do not receive timely responses. Escalation levels progress based on the date of the last outgoing email that necessitated a response. Email notifications are sent to the leader of the user at the previous escalation level, and case details are updated to reflect pertinent escalation information upon successful escalation. Additionally, Agent Super User profiles possess the ability to edit escalation timeframes for optimal case management.

Although the disclosed subject matter has been described in detail for the purpose of illustration based on what is currently considered to be the most practical and preferred embodiments or aspects, it is to be understood that such detail is solely for that purpose and that the disclosed subject matter is not limited to the disclosed embodiments or aspects, but, on the contrary, is intended to cover modifications and equivalent arrangements that are within the spirit and scope of the appended claims. For example, it is to be understood that the presently disclosed subject matter contemplates that, to the extent possible, one or more features of any embodiment can be combined with one or more features of any other embodiment.

What is claimed is:

1. A computer-implemented method, comprising:
diagnosing a condition of a case of a resource planning system using at least one operating parameter;
correlating, by one or more perception nodes of a procurement inference engine, at least one operating parameter with at least one critical parameter of the resource planning system, wherein the one or more perception nodes comprise a neural network trained to generate a prediction of an activity aligned with the at least one critical parameter;
comparing the at least one critical parameter with a predefined threshold value to identify at least one deviation including one or more discrepancies when a critical parameter falls; and
controlling a contract management activity, based on the prediction of the activity aligned with the at least one critical parameter, by generating at least one of a response, a communication, or an action of a contract management system and adapting by automatically adjusting settings or operations to eliminate the at least one deviation to maintain optimal performance.

2. The computer-implemented method according to claim 1, wherein the procurement inference engine includes further instructions, which cause the procurement inference engine to:
diagnose a contract renewal in a first machine learning (ML) model, diagnose a contract maintenance in a second ML model, and diagnose a requisition placement in a third ML model;
diagnose, by the first ML model, an action to take for a contract renewal, wherein one or more inference results are associated with at least one of an initiator, an expiration date, an owner, contract information for a response type associated with at least one of emergency preparedness, a placement, a consignment, a service, a construction, an evaluation, a group purchasing organization (GPO), or a local pricing;
diagnose, by the second ML model, an action to take for a contract maintenance by determining a critical factor associated with a reason for the contract maintenance from at least one of assign to owner, cancelation, current contract, price change, performance notification; and
diagnose, by the third ML model, an assignment of a requisition placement to a commodity.

3. The computer-implemented method according to claim 2, wherein the one or more operating parameters are formed from at least one specified field and are correlated to at least one critical parameter including at least one reason for a contract renewal, a contract maintenance, or a requisition placement.

4. The computer-implemented method according to claim 2, wherein a call to action communication is generated or configured for at least one of an intercompany action, an external action, a response management, or a resolution deployment.

5. The computer-implemented method according to claim 2, comprising:
generating or configuring a call to action based on at least one of: a critical parameter of a cost analysis correlated from operating parameters comprising information about at least one of a usage, a critical parameter of a cost, a critical parameter of an offset, a critical parameter of current product details, or a critical parameter of new product details, a critical parameter of a contract generated with one or more terms, or a critical parameter for one or more identified alternative items,
wherein a critical parameter for the call to action is correlated from a plurality of operating parameters defined by one or more of a current contract identification, a contract description, a supplier name, items on a contract, a start date, an end date, a product name, an item number, a catalog number, a cost, an estimated usage per year, an annualized total cost, product information, a product domain, a product description, a product identification, an alert detail, an alert identification, a release date/type, an alert description, a supplier, an alert reason, a recall level, instructions, additional information, contact information, an inventory item identification, a description, a manufacturer item identification, a vendor item identification, a commodity contract administrator, a clinical resource liaison, a requestor, or a surgery business office manager, and
wherein an agreement type includes at least one of a lease, emergency preparedness, a placement, a consignment, a service, construction, an evaluation, GPO, or local pricing.

6. The computer-implemented method according to claim 5, wherein the call to action comprises:
generating an analysis for at least one of: one or more items of a contract renewal, one or more items of a contract maintenance, or one or more purchase requests of a requisition placement in a plurality of accounts of the resource planning system; and
wherein an analysis of the contract renewal includes at least one of a cost analysis of the usage, a cost analysis of the cost, a cost analysis of an actual spend, an expected contract value (ECV), a probability of the ECV, a contract of indefinite duration, a one-time contract, a quality analysis, a recalls analysis, a rebid for consumer to consumer, or a department approval, one or more contract terms, supplier information, contact information, training determination, source information including minority owned, existing, or new, stocking information, or stocking location,
wherein an analysis of a contract maintenance for an existing agreement includes at least one of an analysis of one or more calls to action of a cancelation, a price change, or a performance notification, and
wherein an analysis of a requisition management for one or more products includes at least one of one or more call to actions, including one or more reason codes, one or more services, one or more items, one or more goods, an existing product, product details, or a source strategy assignment.

7. The computer-implemented method according to claim 6, wherein the procurement inference engine is configured to generate an intercompany action, comprising:
an intercompany action for contract renewal includes automatically generating a call to action for at least one of: a communication to an end user; a contract type, a department approval, determining a renewal, a renew action, a request for proposal, an ad hoc approver, an email generation critical parameter including an operating parameter template, or one or more attachments;
an intercompany action for a contract maintenance for an existing agreement includes at least one of automatically generating a draft agreement based on predicted clauses, at least one department approval, at least one preset commodity groups, at least one ad hock approver, or a contract maintenance use template; and
an intercompany action for a requisition management for one or more products includes determining when a requisition is needed, a call to action operating parameter template, or one or more attachment.

8. A system, comprising:
a memory; and
at least one processor coupled to the memory and configured to:
diagnose a condition of a case of a resource planning system using at least one operating parameter;
correlate, by one or more perception nodes of a procurement inference engine, at least one operating parameter with at least one critical parameter of the resource planning system, wherein the one or more perception nodes comprises a neural network trained to correlate an activity with the at least one critical parameter;
compare the at least one critical parameter with a predefined item threshold value; and
control a contract management activity, based on a prediction of the activity aligned with the at least one critical parameter, by generating at least one of a response, a communication, or an action of a contract management system and adapting by automatically adjusting settings or operations to eliminate at least one deviation to maintain optimal performance.

9. The system according to claim 8, wherein the procurement inference engine includes further instructions, which cause the procurement inference engine to:
diagnose a contract renewal in a first machine learning (ML) model, diagnose a contract maintenance in a second ML model, and diagnose a requisition placement in a third ML model;
diagnose, by the first ML model, an action to take for a contract renewal, wherein one or more inference results are associated with at least one of an initiator, an expiration date, an owner, contract information for a response type associated with at least one of emergency preparedness, a placement, a consignment, a service, a construction, an evaluation, a group purchasing organization (GPO), or a local pricing;
diagnose, by the second ML model, an action to take for a contract maintenance by determining a critical factor associated with a reason for the contract maintenance from at least one of assign to owner, cancelation, current contract, price change, performance notification; and
diagnose, by the third ML model, an assignment of a requisition placement to a commodity.

10. The system according to claim 9, wherein the one or more operating parameters are formed from at least one specified field and are correlated to at least one critical parameter including at least one reason for a contract renewal, a contract maintenance, or a requisition placement.

11. The system according to claim 9, wherein a call to action communication is generated or configured for at least one of an intercompany action, an external action, a response management, or a resolution deployment.

12. The system according to claim 9, comprising:
generating or configuring a call to action based on at least one of: a critical parameter of a cost analysis correlated from operating parameters comprising information about at least one of a usage, a critical parameter of a cost, a critical parameter of an offset, a critical parameter of current product details, or a critical parameter of new product details, a critical parameter of a contract generated with one or more terms, or a critical parameter for one or more identified alternative items,
wherein a critical parameter for the call to action is correlated from a plurality of operating parameters defined by one or more of a current contract identification, a contract description, a supplier name, items on a contract, a start date, an end date, a product name, an item number, a catalog number, a cost, an estimated usage per year, an annualized total cost, product information, a product domain, a product description, a product identification, an alert detail, an alert identification, a release date/type, an alert description, a supplier, an alert reason, a recall level, instructions, additional information, contact information, an inventory item identification, a description, a manufacturer item identification, a vendor item identification, a commodity contract administrator, a clinical resource liaison, a requestor, or a surgery business office manager, and
wherein an agreement type includes at least one of a lease, emergency preparedness, a placement, consignment, a service, construction, an evaluation, GPO, or local pricing.

13. The system according to claim 12, wherein the call to action includes further instructions, which when executed cause the procurement inference engine to:
generate an analysis for at least one of: one or more items of a contract renewal, one or more items of a contract maintenance, or one or more purchase requests of a requisition placement in a plurality of accounts of the resource planning system; and
wherein an analysis of the contract renewal includes at least one of a cost analysis of the usage, a cost analysis of the cost, a cost analysis of an actual spend, an expected contract value (ECV), a probability of the ECV, a contract of indefinite duration, a one-time contract, a quality analysis, a recalls analysis, a rebid for consumer to consumer, or a department approval, one or more contract terms, supplier information, contact information, training determination, source information including minority owned, existing, or new, stocking information, stocking location, and/or the like,
wherein an analysis of a contract maintenance for an existing agreement includes at least one of an analysis of one or more calls to action of a cancelation, a price change, or a performance notification, and
wherein an analysis of a requisition management for one or more products includes at least one of one or more call to actions, including one or more reason codes, one or more services, one or more items, one or more goods, an existing product, product details, or a source strategy assignment.

14. The system according to claim 13, wherein the procurement inference engine includes instructions to generate an intercompany action, comprising:
an intercompany action for contract renewal includes automatically generating a call to action for at least one of: a communication to an end user; a contract type, a department approval, determining a renewal, a renew action, a request for proposal, an ad hoc approver, an email generation critical parameter including an operating parameter template, or one or more attachments,
an intercompany action for a contract maintenance for an existing agreement includes at least one of automatically generating a draft agreement based on predicted clauses, at least one department approval, at least one preset commodity groups, at least one ad hock approver, or a contract maintenance use template; and
an intercompany action for a requisition management for one or more products includes determining when a requisition is needed, a call to action operating parameter template, or one or more attachment.

15. A non-transitory computer-readable medium having instructions stored thereon that, when executed by at least one computing device, cause the at least one computing device to:
diagnose a condition of a case of a resource planning system using at least one operating parameter;
correlate, by one or more perception nodes of a procurement inference engine, at least one operating parameter with at least one critical parameter of the resource planning system, wherein the one or more perception nodes comprises a neural network trained to correlate an activity with the at least one critical parameter;
compare the at least one critical parameter with a predefined item threshold value; and
control a contract management activity, based on a prediction of the activity aligned with the at least one critical parameter, by generating at least one of a response, a communication, or an action of a contract management system and adapting by automatically adjusting settings or operations to eliminate at least one deviation to maintain optimal performance.

16. The non-transitory computer-readable medium according to claim 15, wherein the procurement inference engine includes further instructions, which when cause the procurement inference engine to:
diagnose a contract renewal in a first machine learning (ML) model, diagnose a contract maintenance in a second ML model, and diagnose a requisition placement in a third ML model;
diagnose, by the first ML model, an action to take for a contract renewal, wherein one or more inference results are associated with at least one of an initiator, an expiration date, an owner, contract information for a response type associated with at least one of emergency preparedness, a placement, a consignment, a service, a construction, an evaluation, a group purchasing organization (GPO), or a local pricing;
diagnose, by the second ML model, an action to take for a contract maintenance by determining a critical factor associated with a reason for the contract maintenance from at least one of an assigned owner, cancelation, current contract, price change, performance notification; and
diagnose, by the third ML model, an assignment of a requisition placement to a commodity.

17. The non-transitory computer-readable medium according to claim 15, wherein the one or more operating parameters are formed from at least one specified field and are correlated to at least one critical parameter including at least one reason for a contract renewal, a contract maintenance, or a requisition placement.

18. The non-transitory computer-readable medium according to claim 15, wherein a call to action communication is generated or configured for at least one of an intercompany action, an external action, a response management, or a resolution deployment.

19. The non-transitory computer-readable medium according to claim 15, comprising:
generating or configuring a call to action based on at least one of: a critical parameter of a cost analysis correlated from operating parameters comprising information about at least one of a usage, a critical parameter of a cost, a critical parameter of an offset, a critical parameter of current product details, or a critical parameter of new product details, a critical parameter of a contract generated with one or more terms, or a critical parameter for one or more identified alternative items,
wherein a critical parameter for the call to action is correlated from a plurality of operating parameters defined by one or more of a current contract identification, a contract description, a supplier name, items on contract, a start date, an end date, a product name, an item number, a catalog number, a cost, an estimated usage per year, an annualized total cost, product information, a product domain, a product description, a product identification, an alert detail, an alert identification, a release date/type, an alert description, a supplier, an alert reason, a recall level, instructions, additional information, contact information, an inventory item identification, a description, a manufacturer item identification, a vendor item identification, a commodity contract administrator, a clinical resource liaison, a requestor, or a surgery business office manager, and
wherein an agreement type includes at least one of a lease, emergency preparedness, a placement, a consignment, a service, construction, an evaluation, GPO, or local pricing.

20. The non-transitory computer-readable medium according to claim 19, wherein the call to action comprises:
generating an analysis for at least one of: one or more items of a contract renewal, one or more items of a contract maintenance, or one or more purchase requests of a requisition placement in a plurality of accounts of the resource planning system; and
wherein an analysis of the contract renewal includes at least one of a cost analysis of the usage, a cost analysis of the cost, a cost analysis of an actual spend, an expected contract value (ECV), a probability of the ECV, a contract of indefinite duration, a one-time contract, a quality analysis, a recalls analysis, a rebid for consumer to consumer, or a department approval, one or more contract terms, supplier information, contact information, training determination, source information including minority owned, existing, or new, stocking information, or stocking location,
wherein an analysis of a contract maintenance for an existing agreement includes at least one of an analysis of one or more calls to action of a cancelation, a price change, or a performance notification, and
wherein an analysis of a requisition management for one or more products includes at least one of one or more call to actions, including one or more reason codes, one or more services, one or more items, one or more goods, an existing product, product details, or a source strategy assignment.
